SEQUENCE LISTING (8 sequences)

SEQ ID NO: 1 - DNA, 20 bp, Artificial Sequence
  Description of Artificial Sequence Synthetic primer
acgtcatgatccgctgtcag

SEQ ID NO: 2 - DNA, 23 bp, Artificial Sequence
  Description of Artificial Sequence Synthetic primer
tggatgggagggacagtgcagtg

SEQ ID NO: 3 - DNA, 20 bp, Artificial Sequence
  Description of Artificial Sequence Synthetic primer
ccaggctggtctagaactcg

SEQ ID NO: 4 - DNA, 23 bp, Artificial Sequence
  Description of Artificial Sequence Synthetic primer
tggatgggagggacagtgcagtg

SEQ ID NO: 5 - DNA, 24 bp, Artificial Sequence
  Description of Artificial Sequence Synthetic primer
cacgtgcagccgtttaagccgcgt

SEQ ID NO: 6 - DNA, 25 bp, Artificial Sequence
  Description of Artificial Sequence Synthetic primer
ttcccattctaaacaacaccctgaa

SEQ ID NO: 7 - DNA, 22 bp, Artificial Sequence
  Description of Artificial Sequence Synthetic primer
tctgatgaagtcaggaagaacc

SEQ ID NO: 8 - DNA, 22 bp, Artificial Sequence
  Description of Artificial Sequence Synthetic primer
gagatgtccttcactctgattc

(19) United States
(12) Patent Application Publication
(10) Pub. No.: US 2016/0129021 A1
(43) Pub. Date: May 12, 2016

(54) STERILIZATION METHOD

(71) Applicant: UNIVERSITY OF PITTSBURGH - OF THE COMMONWEALTH SYSTEM OF HIGHER EDUCATION, Pittsburgh, PA (US)

(72) Inventors: William Henry Walker, Pittsburgh, PA (US); Pawan Puri, Pittsburgh, PA (US)

(73) Assignee: UNIVERSITY OF PITTSBURGH - OF THE COMMONWEALTH SYSTEM OF HIGHER EDUCATION, PITTSBURGH, PA (US)

(21) Appl. No.: 14/925,751

(22) Filed: Oct. 28, 2015

Related U.S. Application Data
(63) Continuation of application No. PCT/US2014/034800, filed on Apr. 21, 2014.
(60) Provisional application No. 61/817,133, filed on Apr. 29, 2013.

(51) Int. Cl. A61K 31/655

(52) U.S. Cl. CPC A61K 31/655

(57) ABSTRACT

The present invention relates to methods of decreasing sperm production in a male mammal by administering an effective amount of SHP2 inhibitor to decrease the spermatogonial stem cell (SSC) population. In particular non-limiting embodiments, this method may be used to achieve sterilization. The invention is based, at least in part, on studies in mice which show that (i) in the absence of SHP2, spermatogenesis is blocked at an initial step because spermatogonia cannot be produced from SSCs and (ii) global knock out of SHP2 inhibits the release of mature spermatozoa and causes premature release of germ cells as well as defects in the orientation and migration of elongated spermatids. In certain non-limiting embodiments, the invention provides for a method of decreasing fertility in a male human by administering an effective amount of a SHP2 inhibitor. In other non-limiting embodiments, the invention provides for a method of decreasing fertility in a companion animal such as a dog or cat by administering a SHP2 inhibitor, thereby addressing the problem of overpopulation of these animals.

### PRIORITY CLAIM

This application is a continuation of International Patent Application No. PCT/US2014/034800, filed Apr. 21, 2014, and claims priority to U.S. Provisional Application Ser. No. 61/817,133, filed Apr. 29, 2013, to both of which priority is claimed and the contents of both of which are incorporated herein in their entireties.

### SEQUENCE LISTING

The instant application contains a Sequence Listing which has been submitted electronically in ASCII format and is hereby incorporated by reference in its entirety. Said ASCII copy, created on Dec. 14, 2015, is named 072396.0607_SL.txt and is 2,112 bytes in size.

### 1. INTRODUCTION

The present invention relates to a method for decreasing fertility of human or non-human animals wherein spermatogenesis is reduced by an inhibitor of SHP2 tyrosine phosphatase. In particular embodiments the method may be used as an alternative to surgery for sterilization of companion animals.

### 2. BACKGROUND OF THE INVENTION

### 2.1 Spermatogenesis

Male fertility is maintained by the highly productive process of spermatogenesis that in human males generates more than 100 million sperm every day in the seminiferous tubules of testis.

Spermatogenesis occurs through a similar process across mammalian species. During infancy, gonocyte germ cell precursors give rise to two types of cells: spermatogonial stem cells (SSCs) or differentiated spermatogonia (Bellve et al., 1977; Yoshida et al., 2006). SSCs are rare in adults (for example, SSCs represent only 0.03% of all adult mouse germ cells) and are present as single cells that continuously self-renew and produce undifferentiated spermatogonia that are transit amplifying progenitor cells (Tegelenbosch and de Rooij, 1993). Undifferentiated spermatogonia transition to become differentiated spermatogonia that undergo additional mitotic divisions until a final mitotic division results in a cell committed to meiosis. After completion of meiosis, the haploid round spermatids differentiate into elongated spermatids that are released as spermatozoa when mature.

The correct balance between self-renewal versus differentiation of SSCs is critical to maintain germ cell production and fertility. Continuous differentiation of SSCs into cells committed to sperm development would soon deplete the stem cell pool. Thus, some level of self-renewal is required to preserve stem cells needed to initiate spermatogenesis. Growth factors including GDNF and FGF produced by Sertoli cells support the renewal and proliferation of SSCs (Ishii et al., 2012; Kanatsu-Shinohara et al., 2005; Kubota et al., 2004; Meng et al., 2000; Nagano et al., 2003), but the intracellular factors and signaling pathways that regulate SSC fate decisions are not well characterized.

### 2.2 SHP2

A candidate mediator of GDNF and FGF signaling in SSCs is the widely expressed SHP2 tyrosine phosphatase that is encoded by the Ptpn11 gene. SHP2 is a downstream target of GDNF and FGF. SHP2 also is known to regulate the survival, renewal and proliferation of neuronal, hematopoietic and trophoblast stem cells (Chan et al., 2011; Gauthier et al., 2007; Ke et al., 2007; Yang et al., 2006; Zhu et al., 2011).

Upon growth factor or cytokine stimulation, SHP2 interacts with phosphorylated tyrosine residues on growth factor receptors and stimulates intracellular signaling pathways. SHP2 regulates signaling cascades that are known to decide SSC fate. Specifically, SHP2 stimulates the PI3K/AKT and Ras-MAPK (ERK) pathways but SHP2 can activate or inhibit the JAK/STAT pathway (Grossmann et al., 2010; Kandadi et al., 2010; Xu and Qu, 2008).

Mutations that constitutively activate or inhibit SHP2 activity result in human pathologies. Missense mutations in Ptpn11 that decrease SHP2 activity result in LEOPARD syndrome that is characterized by heart, lung, ocular, growth and genetalia abnormalities (reviewed in (Edouard et al., 2007)). In mice, disruption of the Ptpn11 gene results in embryonic lethality (Arrandale et al., 1996; Saxton et al., 1997; Yang et al., 2006). Constitutive activation of SHP2 can result in juvenile leukemias (Chan and Feng, 2007; Tartaglia et al., 2003) and the juvenile development disorder Noonan Syndrome that includes facial dysmorphia, congenital heart defects, short stature and male infertility (reviewed in (Chan and Feng, 2007; Jorge et al., 2009)).

### 2.3 Companion Animal Overpopulation

Six to eight million cats and dogs enter animal shelters ever year in the United States. Limited shelter resources result in euthanization of approximately half of these animals. There is a great demand for a low cost, single dose sterilant strategy for companion animals to reduce overpopulation.

### 3. SUMMARY OF THE INVENTION

The present invention relates to methods of decreasing sperm production in a male mammal by administering an effective amount of SHP2 inhibitor to decrease the spermatogonial stem cell (SSC) population. In particular non-limiting embodiments, this method may be used to achieve sterilization.

The invention is based, at least in part, on studies in mice which show that (i) in the absence of SHP2, spermatogenesis is blocked at an initial step because spermatogonia cannot be produced from SSCs and (ii) global knock out of SHP2 inhibits the release of mature spermatozoa and causes premature release of germ cells as well as defects in the orientation and migration of elongated spermatids.

In certain non-limiting embodiments, the invention provides for a method of decreasing fertility in a male human by administering an effective amount of a SHP2 inhibitor. In other non-limiting embodiments, the invention provides for a method of decreasing fertility in a companion animal such as a dog or cat by administering a SHP2 inhibitor, thereby addressing the problem of overpopulation of these animals.

### 4. BRIEF DESCRIPTION OF THE FIGURES

FIG. 1A-1J. Induced global knock out of SHP2 blocks spermatogenesis. (A) PCR analysis of testis genomic DNA using primers flanking exon 4 of the gene encoding SHP2. The amplified products are shown for reactions employing genomic DNA isolated from the testes of wild type mice (WT), Ptpn11 floxed mice in the absence of tamoxifen treatment and Ptpn11 floxed mice expressing the Ubc-ert Cre recombinase 29, 43 and 63 days after tamoxifen treatment. (B) Western immunoblot analysis of SHP2 and actin protein expression in whole cell extracts from testes isolated from Ptpn11 floxed mice not treated with tamoxifen (control), SHP2 floxed mice expressing the Ubc-ert Cre recombinase 29 and 43 after tamoxifen treatment. (C-F) Testis tissue sections from wild type mice (C), and Ptpn11fl/fl Ubc-ert2-Cre mice 29 (D), 43 (E) and 63 (F) days after tamoxifen treatment were probed with antisera against VASA (brown stain). Nuclei are stained blue with hematoxylin. Seminiferous tubule cross sections showing disrupted spermatogenesis as measured by two or fewer layers of germ cells are marked with an asterisk (*). Magnification=20×. (G-J) Testis tissue sections from wild type mice (G), and Ptpn11fl/fl Ubc-ert2-Cre mice 29 (H), 43 (I) and 63 (J) days after tamoxifen treatment were stained with periodic acid Schiffs-hematoxylin (PAS-H). Sertoli cell nuclei are marked with black arrowheads. Spermatogonia and preleptotene spermatocytes on the basement membrane are marked with green arrows. Vacuoles in the regions lacking germ cells are noted with red arrows. The red oval marks a region on the basement membrane lacking spermatogonia. Mis-localized elongated spermatids are marked with orange arrows. Magnification: C-F=20×, Bar=100 microns, G-J=60×, bar=20 microns.

FIG. 2A-D. SHP2 is expressed in spermatogonial stem cells and undifferentiated spermatogonia. (A) Adult wild type mouse testis sections were probed with antiserum against SHP2 and stained with hematoxylin (blue). The SHP2 immunostaining (brown) was localized to Sertoli (S) nuclei and Sertoli cell cytoplasm between germ cells (black arrowheads) and around elongated spermatids (ES). SHP2 immunostaining was also present along the basement membrane in a region consistent with the blood testis barrier (red arrows) that did not extend beyond the apical side of spermatogonia (Sp) or preleptotene spermatocytes (P1). Bar=20 microns. (B) P15 wild type mice were probed with antisera against PLZF (top, red cells) or antisera against SHP2 (bottom, green). Nuclei were stained blue with Dapi. Bar=100 microns. Cells along the basement membrane are numbered for comparisons. (C) Images of adult testis cross sections are shown after probing with antisera against PLZF and SHP2 and the merging of the two signals. Arrows mark spermatogonia that express PLZF and SHP2. Bar=20 microns. (D) Images of GS cells probed with PLZF and SHP2 and the merged signals. White arrows mark clusters of GS cells that express PLZF and SHP2. Red arrows mark larger nuclei of feeder cells in the background that express SHP2. Bar=20 microns.

FIG. 3. Undifferentiated and differentiated spermatogonia are not present after global knock out of SHP2. Testis tissue from Ptpn11fl/fl mice lacking cre recombinase (Control) and Ptpn11^{Δ/Δ} mice 29, 43, and 63 days after tamoxifen treatment were probed with antisera against PLZF, SOHLH1 and GATA 4. Representative immunopositive staining (brown) is noted with arrows. Magnification=60×. Bar=20 microns.

FIG. 4A-E. Ablation of SHP2 expression in germ cells results in the loss of all germ cells. (A) PCR analysis of the region encompassing exon 4 of Ptpn11 in testis tissue from wild type (WT), floxed Ptpn11 (floxed), GCSHP2KO mice and mice heterozygous for floxed Ptpn11 (Heterozygous). Analysis of Cre expression from the VasaCre transgene is shown below. (B) Western immunoblot analysis of whole testis extracts from wild type and GCSHP2KO (KO) mice probed with antisera against SHP2 or β-actin. (C, D) Mean (+SEM) weights of a pair of testes (C) and seminiferous tubule diameters (D) from 3-, 4- and 8-week old control and KO mice are shown. Age matched pairs marked with asterisks (*) differ significantly (p [|t]0.05). For (C) n=2 except 8 week-old n=5. For (D) at least 10 cross-sections were measured for each condition. (E) Testis tissue from 3, 4 and 8 week-old control and GCSHP2KO mice stained with periodic acid-Schiff-hematoxyin. Magnification=20×. Bar=100 microns. Inserts show the relative sizes of the testes isolated from wild type and GCSHP2KO mice.

FIG. 5. Undifferentiated and differentiated spermatogonia are not produced by GCSHP2 mice. Testis tissue sections from 3 week-old control and 3, 4 and 8 week old GCSHP2KO mice were probed with antisera against Vasa, SOHLH, PLZF and GATA4. For Vasa staining, arrows mark the presence of stained spermatogonia on the basement membrane (Control testes only) and more mature germ cells in the adluminal compartment in control and GCSHP2 testes. For SOLHLH1 and PLZF staining, arrows mark differentiated and undifferentiated spermatogonia, respectively present only in control testes. For GATA4 staining, arrows mark Sertoli cell nuclei in all conditions. Magnification=60×. Bar=20 microns.

FIG. 6A-H. The number of SSCs and undifferentiated spermatogonia are decreased five and seven days after birth in GCSHP2KO mice. (A) Testis tissue sections from P2 control and GCSHP2KO mice were probed with antisera against Vasa or GATA4. (B), (C) Testis sections from P5 and P7 control and GCSHP2KO mice were probed with PLZF or GATA4 antisera. (D) Quantitation of Vasa positive (P2, n=1) and PLZF positive (P5, n=4 and P7, n=2) cells per tubule for P2, P5 and P7 control and GCSHP2KO testis sections. (E) Quantitation of TUNEL positive cells per tubule for P2 (n=1), P5 (n=2) and P7 (n=2) control and GCSHP2KO testis sections. (F) Testes sections from P5 and P7 control and GCSHP2KO mice were probed with antisera against PLZF and the apoptosis marker cleaved caspase-3. The percentage of PLZF-positive cells that also express cleaved caspase-3 was quantified from entire tissue sections (259-1,453 PLZF cells/section, n=3). (G) Testis sections from P5 and P7 control and GCSHP2KO mice were probed with antisera against PLZF and the proliferation marker Ki-67. The percentage of PLZF-positive cells that also express Ki-67 was quantified as in panel G from 386 to 1,107 PLZf-positive cells/section. Statistical analysis of P5 control versus P5 GCSHP2KO as well as P7 control versus P7 GCSHP2KO comparisons in panels F and G was evaluated by unpaired t test. Asterisks denote pairs in which the means are statistically significant (n=3). (H) Schematic summary of spermatogenesis including gonocytes producing differentiated A2 spermatogonia in the first wave. After conversion of gonocytes to SSCs, successive waves of spermatogenesis require SSC self-renewal and production of A aligned (Aal) undifferentiated spermatogonia that transition into differentiated spermatogonia and eventually to sperm.

FIG. 7A-F. Inhibition of SHP2 blocks GDNF and FGF signaling causes SSCs to detach from their niche decreases the number of SSCs in culture and disrupts spermatogenesis in vivo. (A) GS cultures were stimulated for 30 min with FGF (20 ng/ml) or GDNF 20 ng/ml) with or without 5 h pretreatment with NSC-87877 (NSC, 50 μM). Western blot analysis of p-ERK and ERK levels after the treatments is shown. (B) GS cultures were treated for 5 days with vehicle or NSC-87877 (50 μM). The percentage of cells detached from feeder cells was determined (n=3, *=p<0.05). C) The epididymis of control and GCSHP2KO mice were stained with periodic acid Schiffs-hematoxylin. Immature germ cells in GCSHP2KO epididymis are identified with arrows. D) GS cells treated with vehicle or NSC (50 μM) as in B were isolated from feeder cells, dispersed and counted. E) Clumps of GS cells (white arrows) and feeder cells (yellow arrows) treated as in D were assayed for live (green) and dead (red) cells. (F) Testis sections from wild type mice isolated 15 days after injection with vehicle, or NSC-87877 for 3 or 7 days were stained with PAS-H. Green arrows: spermatogonia, black arrows: Sertoli cell nuclei, Orange arrows: mis-localized elongated spermatids, Blue arrow: a cluster of prematurely detached germ cells.

FIG. 8. Immature germ cells are released prematurely after SHP2 knock out. Cross sections of epididymis from SHP2flox/flox lacking cre recombinase (Control) and Ptpn11fl/fl Ubc-ert2-Cre mice 63 days after tamoxifen treatment were probed with antisera against VASA or SOHLH1 (arrows, brown staining). Nuclei are stained blue with hematoxylin.

FIG. 9. SHP2 immunostaining is absent in all germ cells and some Sertoli cells in Ptpn11^{Δ/Δ} mice. Control and Ptpn11^{Δ/Δ} mice testis cross-sections probed with SHP2 antisera (top, red) and stained with Dapi (middle, blue) and merged signals (bottom). Sertoli (S) cell nuclei with immunoreactive SHP2 are denoted with white arrows. Sertoli nuclei without SP2 immunostaining are denoted with red arrows. Spermatogonia (Sp) are denoted with white arrowheads. Leptotene (L) and pachytene (P) spermatocytes round spermatids (Rs) and elongated spermatids (Es) are also identified with white arrows.

FIG. 10. One injection of the SHP2 inhibitor NSC-87877, at a dose of 40 mg/kg, into the testes of adult mice was sufficient to disrupt spermatogenesis (sperm production) 15 days later.

FIG. 11. One injection, at a dose of 10 mg/kg, of NSC-87877 into the testes of adult mice produced long term (70 days) loss of sperm production in some regions of the testes, Asterisks denote regions of the testes lacking germ cells and/or sperm.

FIG. 12 shows that spleen tissue from the same animal of FIG. 11 was not damaged 15 days after injection of the inhibitor into testes.

### 5. DETAILED DESCRIPTION OF THE INVENTION

For clarity, and not by way of limitation, the detailed description of the invention is divided into the following subsections:

(i) SHP2 inhibitors;

(ii) subjects;

(iii) methods; and

(iv) compositions.

### 5.1 SHP2 Inhibitors

A SHP2 inhibitor for use according to the invention decreases SHP2 activity, and would include agents that decrease SHP2 expression (e.g. antisense, small interfering, and/or hairpin RNA that comprises a region complementary to SHP2 RNA) as well as those that inhibit the function of the SHP2 molecule. Non-limiting examples of SHP2 inhibitors include NSC-87877 (also known as 8-Hydroxy-7-[(6-sulfo-2-naphthyl)azo]-5-quinolinesulfonic acid), having the following structure:

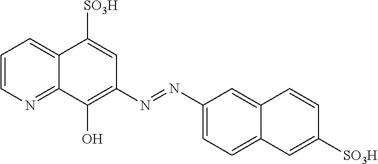 [SMILES: O=S(=O)(O)c1ccc2cc(/N=N/c3cc(S(=O)(=O)O)c4cccnc4c3O)ccc2c1]

estradiol phosphate, estramustine phosphate, PHPS1, NSC-117199, SP1-112, SP1-112Me (and see Chen, L. et al., 2006 and Chen, L. et al., 2010), tautomycetin analogs (e.g., see Liu, S. et al., 2011), phenylhydrazonopyrazolone sulfate and compounds described in Hellmuth, K. et al., 2008, compounds described in United States Patent Application Publication No. 20120034186 (U.S. Ser. No. 13/274,699) and compounds described in Yu, Z. H. et al. 2011.

### 5.2 Subjects

The present invention may be applied to mammalian subjects who would benefit from a decrease in fertility or for whom a decrease in fertility would otherwise be desirable. For example, and not by way of limitation, the subject may be a human or a non-human animal such as a dog, cat, mouse, rat, rabbit, skunk, hamster, guinea pig, groundhog, prairie dog, beaver, coyote, bear, deer, cattle, pig, horse, goat, etc. In one specific, non-limiting embodiment, the subject may be a feral cat.

### 5.3 Methods

In certain non-limiting embodiments, the invention provides for a method of reducing fertility in a subject comprising administering, to the subject, an effective amount of a SHP2 inhibitor.

In certain non-limiting embodiments, the invention provides for a method of reducing spermatogesis in a subject comprising administering, to the subject, an effective amount of a SHP2 inhibitor.

An effective amount of a SHP2 inhibitor is an amount which results in one or more of the following: a significant decrease in SHP2 activity in a spermatogonial sperm cell in the subject; a significant decrease in SHP2 activity in a Sertoli cell in the subject; a significant decrease in the number of spermatogonial stem cells in the subject; and/or a significant decrease in the sperm cell count in an ejaculate of the subject. A significant decrease may be at least about 20 percent or at least about 30 percent or at least about 50 percent. These results may be measured after an interval to allow the inhibitor to act on SHP2 and/or have consequences on the SSC and/or mature sperm cell population (see, for example, the working example below).

As SHP2 inhibitors are used to reduce the number of SSCs, the decrease in fertility may be essentially irreversible. Dosage of SHP2 inhibitor may be adjusted so that the decrease in fertility may be at least partially reversible.

The SHP2 inhibitor may be administered according to methods known in the art, including but not limited to by one or more of the following routes: oral, intravenous, intraperitoneal, intramuscular, subdermal, or by local injection/surgical introduction into the testicle. In one non-limiting embodiment, the SHP2 inhibitor may be administered via a sustained release implant.

In certain non-limiting embodiments, the SHP2 inhibitor may be administered to a subject prior to sexual maturity. In other non-limiting embodiments, the SHP2 inhibitor may be administered to a subject after sexual maturity has been achieved. In certain non-limiting embodiments where the subject is a dog or cat, the SHP2 inhibitor may be administered when the animal is at least four weeks old, or between one and eight months of age, or between two and six months of age, or between four and eight months of age, or between four and six months of age.

In certain non-limiting embodiments, the SHP2 inhibitor may be administered either once or multiple times, for example at least once, at least twice, at least three times, at least four times, in single or divided doses. In certain non-limiting embodiments, the SHP2 inhibitor may be administered repeatedly, for example, about twice a year, or about once a year, or about once every two years, or about once every three years. In a certain non-limiting embodiment, the SHP2 inhibitor may be administered to a companion animal once a month for four consecutive months

After administration of the SHP2 inhibitor, it may be desirable to temporarily attempt to limit exposure of the subject to infectious agents

In certain non-limiting embodiments, the dosage of SHP2 inhibitor may be a dose of SHP2 inhibitor which would be about equipotent (referring to SHP2 inhibition) to (i) between about 0.1-100 mg/kg of NSC-87877 or (ii) between about 0.2 and 50 mg/kg of NSC-87877 or (iii) between about 0.05 and 10 mg/kg of NSC-87877 or (iv) between about 0.1 and 5 mg/kg of NSC-87877; or (v) between about 1 and 100 mg/kg of NSC-87877. In certain specific non-limiting embodiments, the dosage of SHP2 inhibitor may be (i) between about 0.1-100 mg/kg of NSC-87877 or (ii) between about 1 and 50 mg/kg of NSC-87877 or (iii) between about 0.05 and 10 mg/kg of NSC-87877 or (iv) between about 0.1 and 5 mg/kg of NSC-87877 or (v) between about 1 and 100 mg/kg of NSC-87877 or between about 1 and 20 mg/kg of NSC-87877. In specific non-limiting embodiment, the above doses may be administered to a subject that is a human, a dog, or a cat. As one non-limiting example, as illustrated by working example 2 below, a single dose of 10 mg/kg or 40 mg/kg administered to a mouse was effective in decreasing fertility.

In certain non-limiting embodiments, the dosage of SHP2 inhibitor may be a dose of SHP2 inhibitor which would produce a local concentration of inhibitor in the testes of the subject which would be about equipotent (referring to SHP2 inhibition) to between about 0.5 to 200 μg/ml, or between about 1-100 μg/ml, or between about 10 and 80 μg/ml, or between about 30 and 80 μg/ml, or about 50 μg/ml, of NSC-87877. In certain specific non-limiting embodiments, the dosage of SHP2 inhibitor may be a dose of NSC-87877 which would produce a local concentration of NSC-87877 in the testes of the subject which would be between about 0.5 to 200 μg/ml, or between about 1-100 μg/ml, or between about 10 and 80 μg/ml, or between about 30 and 80 μg/ml, or about 50 g/ml.

### 5.4 Compositions

In certain non-limiting embodiments, where the SHP2 inhibitor is to be administered orally, the SHP2 inhibitor may be comprised in a palatable form, for example, a tablet comprising a dose of SHP2 inhibitor, one or more tableting excipients, and optionally flavoring for example beef, chicken, fish, cheese, liver, or peanut butter flavoring.

In a specific non-limiting embodiment, the SHP2 inhibitor in such a tablet may be NSC-87877, for example, in an amount between about 0.01 mg-5 g, or between about 0.1 mg and 2 g, or between about 0.1 and 100 mg, or between about 1 and 100 mg, or between about 10 and 200 mg, or between about 20 and 400 mg, or between about 50 mg and 1 g In alternate non-limiting embodiments the tablet may comprise about an equipotent amount (in terms of SHP2 inhibitor activity) of another SHP2 inhibitor. In another non-limiting embodiment, the SHP2 inhibitor may be administered as a dry powder, for example which is added to a portion of food (e.g., an effective dose of SHP2 inhibitor comprised in a pharmaceutical powder). In another non-limiting embodiment, an effective dose of SHP2 inhibitor may be comprised in a food product, for example a kibble or pellet-type dietary preparation.

Where a SHP2 inhibitor is to be administered by injection, the SHP2 inhibitor may be provided in dry form, for example, in a sterile vial, and then a pharmaceutically appropriate liquid may be added prior to injection, or the SHP2 inhibitor may be provided in a suitable pharmaceutical liquid form.

### 6. EXAMPLE 1

### The Protein Tyrosine Phosphatase SHP2 is Essential for Self-Renewal of Spermatogonial Stem Cells

### 6.1 Materials and Methods

Animal Care and Use.

Previously described Ptpn11^{fl/fl} mice and Ubc-ert2-Cre transgenic mice (Ruzankina et al., 2007; Zhang et al., 2004) were mated to generate Ptpn11^{fl/fl} ert2-Cre mice. To create Ptpn11^{Δ/Δ} mice, the Ptpn11^{fl/fl} ert2-Cre mice were injected twice with tamoxifen (Sigma: 200 μg/g body weight in corn oil on 2 consecutive days at 6 to 8 weeks of age as described (Bauler et al., 2011) and sacrificed 29, 43 or 63 days later. To generate GCSHP2KO mice, Ptpn11^{fl/fl} mice were mated with Vasa-Cre (Ddx4-cre) mice (obtained from Dr. A. Rajkovic, Univ. of Pittsburgh). Stra8SHP2KO mice were produced by the mating of previously described Stra8-cre mice (Sadate-Ngatchou et al., 2008) with Ptpn11^{fl/fl} mice. Generation and genotypic identification of ptpn11^{fl/fl} mice and ubiquitin-promoter-driven ert2-cre transgenic mice has been described elsewhere (Ruzankina et al., 2007; Zhang et al., 2004). Animals used in these studies were maintained and euthanized according to the principles and procedures described in the NIH Guide for the Care and Use of Laboratory Animals. These studies were approved by the University of Pittsburgh Institutional Animal Care and Use Committee.

Reagents and Antibodies.

FGF, GDNF and NSC-87877 were obtained from BD Biosciences (San Jose, Calif.), Peprotech (Rocky Hill, N.J.), Tocris Bioscience (Bristol, U.K.), respectively. Antisera applications and the dilutions used are summarized in Table 1.

TABLE 1
Antibody applications and dilutions

| Antibody | Company | Catalog # | Application | Dilution |
|---|---|---|---|---|
| SHP2 | Santa Cruz | SC-280 | Western Blotting | 1:1000 |
| | Biotechnology | | Immunofluorescence | 1:2000 |
| | | | Immunohistochemistry | 1:2000 |
| pERK | Cell Signaling | 9101s | Western Blotting | 1:1000 |
| ERK | Upstate | 06-182 | Western Blotting | 1:20000 |
| □-actin | Sigma | A5316 | Western Blotting | 1:10000 |
| Vasa | Abcam | Ab13840 | Immunohistochemistry | 1:2000 |
| SOHLH1 | (Suzuki et al., | N/A | Immunohistochemistry | 1:1000 |
| | 2012) | | | |
| PLZF | R & D Systems | AF2944 | Immunofluorescence | 1:500 |
| | | | Immunohistochemistry | 1:500 |
| GATA4 | Santa Cruz | SC-1237 | Immunohistochemistry | 1:500 |

Preparation of Whole Cell Extracts and Western Blot Analysis.

Whole cell lysates (WCE), were isolated from 28 to 60 day-old control and GCSHP2KO mouse testes analyzed by western blot as previously described (Puri and Walker, 2013).

GS Cultures.

Isolation of THY1+ germ cells from 6-day-old DBA/2 mice was performed as described previously to produce GS cell cultures (Oatley and Brinster, 2006) (more details in Supplemental Experimental Procedures). GS cultures were starved of GDNF and FGF overnight and incubated in the absence and presence of NSC-87877 for 6 to 8 hrs. SSCs were detached from feeders and FGF or GDNF were added for 10 min. Detached SSCs were then pelleted (600×g, 7 min) and the pellets were lysed in Laemmli sample buffer.

Immunocytochemistry and Immunofluorescence.

Immunostaining of testis tissue was performed on paraffin-embedded sections (5 m) from paraformaldehyde (4%, o/n) or Bouins fixed adult rat testis as previously described (Puri and Walker, 2013). The testis tissue or cultured cells were then incubated 12-24 h with preimmune serum or rabbit polyclonal antiserum directed against specific antisera as detailed in Table 1. Colorimetric and fluorescent detection of immune complexes were performed as previously described (Puri and Walker, 2013). A charged coupled device (CCD) video camera system was used to capture images of stained cells or tubule cross-sections. All files were digitally processed fro presentation using Adobe Photoshop (Adobe Systems, Inc).

TUNEL Assay, Cell Counting and LIVE/Dead Staining.

Paraformadehyde fixed testis sections were evaluated using a TUNEL assay kit (Roche).

All immunostained cells and tubules from an entire testis section were counted and the number of stained cells per tubule was determined. GS cell viability was determined using the LIVE/DEAD Viability Assay kit for mammalian cells (Life Technologies)

Statistical Analysis.

Immunoreactive signals from western blot films were scanned with an Epson 1600 Expressions scanner using Epson Scan soft ware. For western blots, the mean±SEM relative signal intensities were determined for at least three independent experiments. Results were analyzed by ANOVA with Newman-Keuls PLSD at a 5% significance level utilizing GraphPad Prism 4.3 (GraphPad Software).

Genotyping.

Identification of floxed Ptpn11 alleles was performed as previously described using a forward primer in exon 4 (5′-ACG TCA TGA TCC GCT GTC AG-3′; SEQ ID NO: 1) and a reverse primer in intron 4 (5′-TGG ATG GGA GGG ACA GTG CAG TG-3′; SEQ ID NO:2) (Zhang et al., 2004). The presence of the recombined Ptppn11 allele was determined by polymerase-chain-reaction using primers complementary to the intron 3 (5′-CCA GGC TGG TCT AGA ACT CG-3′; SEQ ID NO:3) and intron 4 (5′-TGG ATG GGA GGG ACA GTG CAG TG-3′; SEQ ID NO:4) sequences of the Ptpn11 gene. The Vasa Cre transgene was detected by using primers complimentary to the Vasa Promoter (5′-CACGTGCAGCCGTTTAAGCCGCGT 3′; SEQ ID NO:5) and globin intron (5′-TTCCCATTCTAAACAACACCCTGAA; SEQ ID NO:6). Stra8 mice were genotyped by using primers i-Cre-F (5′-TCTGATGAAGTCAGGAAGAACC; SEQ ID NO:7) and i-Cre R (5′-GAGATGTCCTTCACTCTGATTC; SEQ ID NO:8).

Preparation of Whole Cell Extracts and Western Blot Analysis.

To prepare testis whole cell lysates (WCE), testes isolated from 28 to 60 day-old control and GCSHP2KO mice were decapsulated and homogenized in enhanced lysis buffer (ELB) (250 mM NaCl, 0.1% NP40, 50 mM Hepes, pH 7.0, 5 mM 132 EDTA, 0.5 mM dithiothreitol) with a protease inhibitor cocktail rocked for 15 min at 4° C. and then pelleted (12,000×g 15 min) to remove cell debris.

WCE boiled in Laemmli sample buffer were fractionated by 10% SDS-PAGE and transferred to Immobilon-P, PVDF membranes 90 (Millipore Corp., Bedford, Mass.). Nonspecific binding sites were blocked with 5% non fat dry milk in Tris-buffered saline (TBS; 25 mM TRIS-HCl, pH 7.4, 0.15 M NaCl, containing 0.1% Tween 20) followed by incubation overnight at 4° C. with the appropriate primary antibody against SHP2, p-ERK, ERK, or β-Actin. The blots were washed and incubated for 1 h at room temperature with anti-rabbit raised in donkey (1:20,000) for primary antibodies against SHP2, p-ERK and ERK or anti-mouse antibodies raised in donkey (1:10,000) for actin antiserum. The antigen-antibody complex was visualized with chemiluminescent HRP substrate (Millipore Corp.)

GS Cultures.

Preparation of GS cell cultures from 6-day-old DBA/2 mice was performed as described previously (Oatley and Brinster, 2006). Briefly, a testicular cell suspension was isolated by enzymatic digestion with DNase and trypsin and filtered through a 40 uM mesh to remove cell clumps. Germ cells were then enriched from these suspensions through a 30% percoll gradient. The germ cell suspensions were subjected to magnetic activated cell sorting (MACS) to select Thy1+ cells to enrich for SSCs. The cells were washed in mouse SSC serum-free medium (mSFM) and plated onto STO feeders in mSFM supplemented with 20 ng/ml recombinant human GDNF and 20 ng/ml recombinant FGF2 (FGF). GS cultures were maintained in these conditions at 37° C. in an atmosphere of 5% CO2 in air and subcultured at 1:2 to 1:3 ratios onto fresh STO feeders every 7 days. Primary cultures were used for experiments between 1 and 3 mo after establishment.

Additional Details for Immunocytochemistry and Immunofluorescence.

Testis sections were deparaffinized in xylene and rehydrated. The sections were subjected to antigen retrieval in citrate buffer (10 mm citrate, pH 6.0, containing 0.1% Tween-20) at 95° C. for 30 min and then left undisturbed at room temperature for 30 min. The sections were washed two times for 5 min in PBS and blocked for 1 h in goat or donkey or horse serum at room temperature. The testis sections were then incubated 12-24 h with pre-immune serum or antiserum SHP2, Vasa, SOHLH1, PLZF and GATA4. Cultured cells were fixed in 4% paraformaldehyde for 5 min, permeablized for 1 min in ice-cold 100% MeOH, and dried completely followed by blocking with normal goat serum, 0.5% BSA, and 0.15% glycine. For colorimetric assays, anti-rabbit or anti mouse biotinylated secondary antibody (Vectastain Elite ABC Kit, Vector Laboratories) was added, and bound antibodies were detected using DAB staining colorimetric reagent and counterstained with hematoxylin. For immunofluorescence studies, Cy3 and ALexa488 conjugated secondary antisera were added, nuclei were stained with 4′6′-diamidino-2-phenylindole (DAPI) and immunostaining was detected using a Nikon Provis II fluorescence microscope or confocal microscope.

### 6.2 Results

The Induced Global Knock Out of SHP2 in Adult Mice Blocks Spermatogenesis.

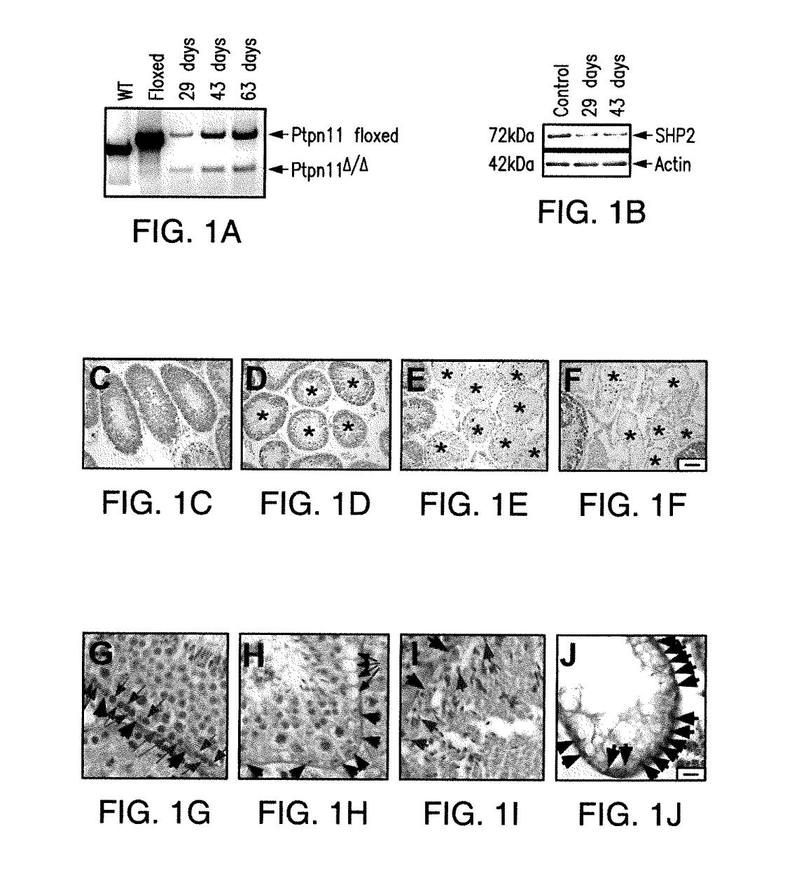

To define the function of SHP2 in spermatogenesis, we first eliminated SHP2 expression in adult Ptpn11^{fl/fl} Ubc-ert2-Cre mice. These mice express a tamoxifen inducible CRE recombinase driven by a ubiquitin promoter. Administration of tamoxifen to these mice causes the excision of exon 4 from the Ptpn11 gene, which results in the introduction of a stop codon and a truncated, inactive SHP2 protein (Bauler et al., 2011; Zhang et al., 2004). Adult (>60 days-old) Ptpn11^{fl/fl} Ubc-ert2-Cre and Ptpn11^{fl/fl} (control) mice were treated for 2 days with tamoxifen to ablate SHP2 expression and sacrificed 29, 43 and 63 days later. PCR analysis of the DNA isolated from the testis of the tamoxifen treated Ptpn11^{fl/fl} Ubc-ert2-Cre mice (hereafter Ptpn11^{fl/fl} mice) and control mice confirmed the Cre-mediated excision of Ptpn11 exon 4 in the testis (FIG. 1A). Western blot analysis showed that the protein levels of SHP2 in animals treated with tamoxifen were decreased (FIG. 1B). The PCR and western blot analyses also indicated that the excision of exon 4 does not occur in all cells and that some SHP2 protein expression is retained in the Ptpn11^{Δ/Δ □□ mouse testis.}

Testes weights of Ptpn11^{Δ/Δ} mice 29 and 43 days after tamoxifen treatment were reduced by 36% and 52%, respectively. In contrast to the intact spermatogenesis in control mice (FIG. 1C), spermatogenesis was disrupted in 33% of tubule cross sections from Ptpn11^{Δ/Δ} mice 29 days after tamoxifen treatment as defined by the presence of two or fewer layers of germ cells stained with Vasa antiserum that stains all germ cells (FIG. 1D). At the 43 and 63 day time points, 43% and 27% of tubule cross sections showed disrupted spermatogenesis, respectively (FIGS. 1E, 1F). The mosaic nature of the gene knockout is commonly seen in tamoxifen induced knockout mouse models (Metzger and Chambon, 2001) and varying efficiencies of gene ablation using the Ubc-ert2-Cre to knock out SHP2 has been shown for various tissues (Bauler et al., 2011). The presence of tubules with intact spermatogenesis also indicates that the observed disrupted spermatogenesis in affected tubules did not result from nonspecific actions of tamoxifen, damage to Leydig cells or some insult to the hypothalamic-pituitary-testis endocrine axis.

Knock Out of SHP2 Blocks Spermatogenesis Prior to Spermatogonia Development and Inhibits Germ Cell Attachment to Sertoli Cells.

The severe disruption of spermatogenesis in Ptpn11^{Δ/Δ} mice led us to examine the germ cell complement of seminiferous tubules from Ptpn11^{Δ/Δ} testes in greater detail. In comparison to testes from control mice (FIG. 1G), spermatogonia and preleptotene spermatocytes were absent from the basement membrane of disrupted tubules Ptpn11^{Δ/Δ} mice 29 days after tamoxifen treatment (FIG. 1H). Leptotene and zygotene spermatocytes also were absent from some tubules. Numerous vacuoles were found in the disrupted tubules in locations in which premieotic and meiotic germ cells were missing. Pachytene spermatocytes as well as round and elongated spermatids were present 29 days after tamoxifen treatment. Many of the elongated spermatids were mis-localized near the basement membrane in stage VI-IX tubules suggesting that they were not able to migrate to the lumen to be released. This mis-localization of elongated spermatids has been shown to result in phagocytosis of the germ cells by Sertoli cells (Russell et al., 1989).

At 43 days after knock out of SHP2, all germ cells were absent except for elongated spermatids (FIG. 1I). The elongated spermatids were distributed randomly with many having their acrosomes mis-oriented away from the Sertoli cell nuclei suggesting that the attachment of the elongated spermatids to the supporting Sertoli cells was mis-regulated. Because the time required to complete spermatogenesis in the mouse is 35 days and because germ cells were not replenished after SHP2 knock out, the presence of elongated spermatids 43 days after SHP2 knock out suggests that the release of elongated spermatids from Sertoli cells is inhibited. Furthermore, the localization of many elongated spermatids near the basement membrane again is consistent with phagocytosis of the elongated spermatids by Sertoli cells. At 63 days after tamoxifen treatment, tubules with disrupted spermatogenesis contained only Sertoli cells as all developmental stages of germ cells were absent (FIG. 1J). This result confirmed that germ cells are not replenished after knock out of SHP2.

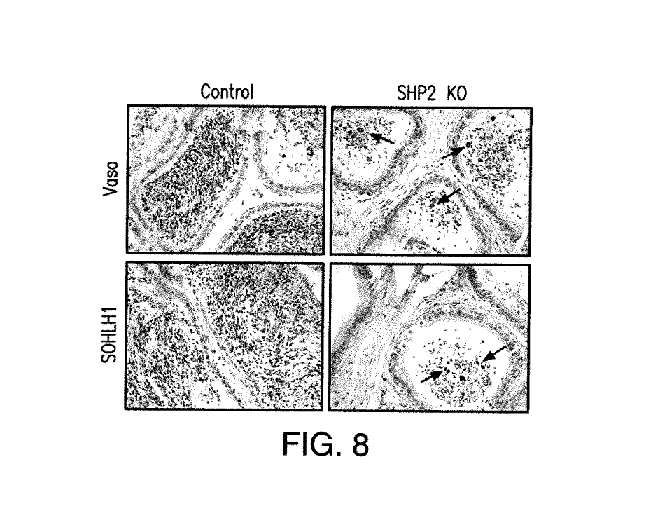

Ptpn11^{Δ/Δ} male mice retained normal mating behavior but fertility was reduced as matings of at least 6 weeks produced litters at a 50% rate (5/10); whereas, control mice treated with tamoxifen produced litters at a rate of 82% (14/17). Fertility of the SHP2 knock out mice is likely maintained due to the presence of tubules in which SHP2 expression was retained and spermatogenesis was not disrupted. Probing of epididymis tissue cross sections from Ptpn11^{Δ/Δ} mice confirmed that sperm were present. However, many epididymis cross sections lacked sperm 63 days after SHP2 knock out; whereas, other cross sections had reduced numbers of sperm plus prematurely detached germ cells (FIG. 8). Immunostaining with antisera against Vasa or the SOHLH1 transcription factor, which is a marker for differentiated spermatogonia (Suzuki et al., 2012) revealed that the prematurely released germ cells present in the epididymis after SHP2 knock out included spermatogonia, spermatocytes and round spermatids. The presence of immature germ cells in the epididymis suggests that there are defects in Sertoli cell-germ cell attachment in the Ptpn11^{Δ/Δ} mice, which is consistent with earlier findings that constitutive activation of SHP2 resulted in mis-localization of adhesion related proteins in Sertoli cells (Puri and Walker, 2013).

SHP2 is Expressed in SSCs and Undifferentiated Spermatogonia.

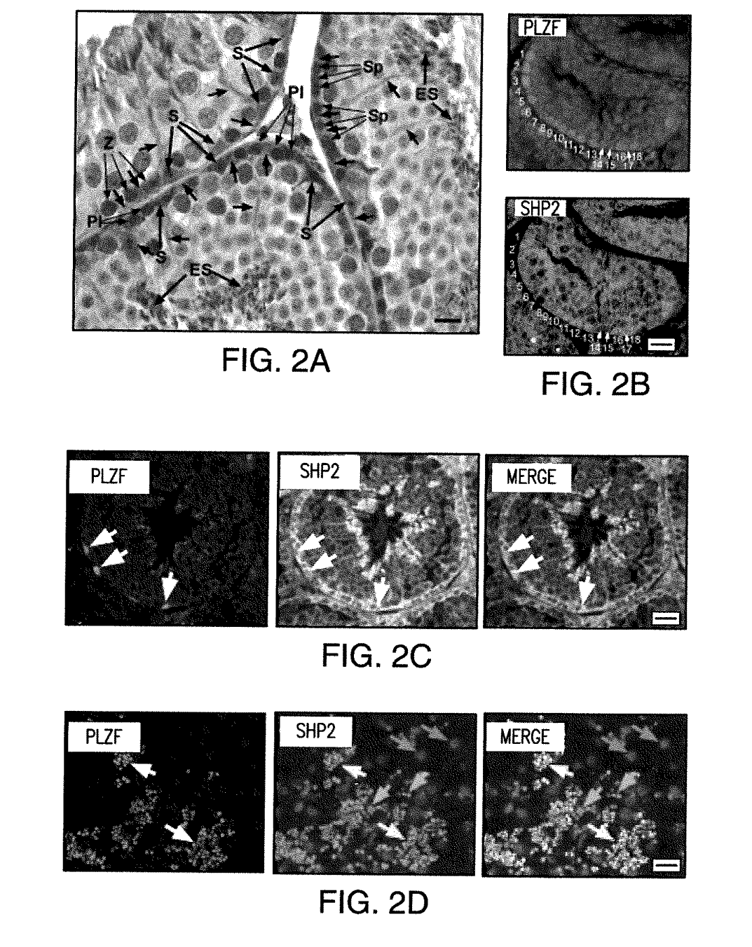

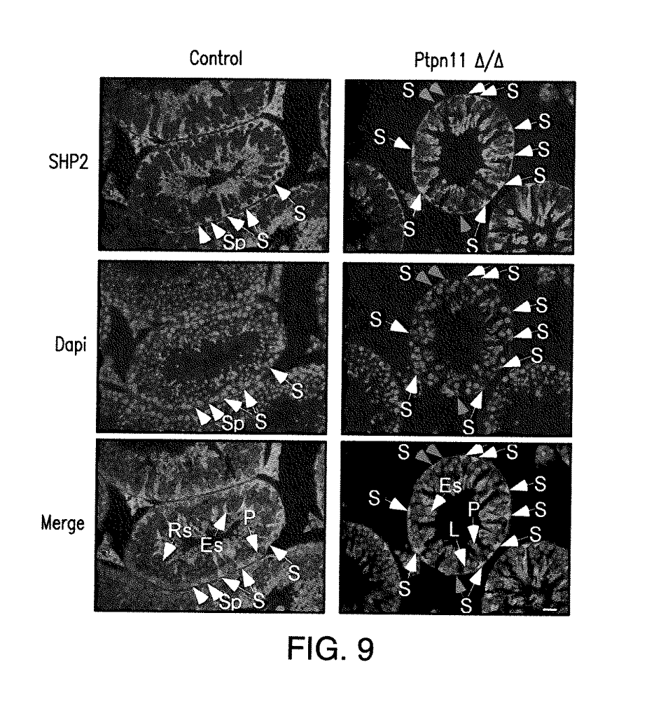

Immunohistochemical studies of adult mouse testis sections determined that SHP2 was expressed in Sertoli cell nuclei as well as in Sertoli cytoplasm at the basement membrane, the blood testis barrier (BTB) and between germ cells (FIG. 2A). SHP2 also was expressed in spermatogonia on the basement membrane but expression was low or undetectable in spermatocytes and spermatids. Strong SHP2 immunostaining in Sertoli cells was localized surrounding maturing elongated spermatids that is consistent with attachment sites between Sertoli cells and elongated spermatids. In Ptpn11^{Δ/Δ} mice 29 days after tamoxifen treatment, spermatogonia were not present and SHP2 was not detected in any of the remaining germ cells in the tubules with disrupted spermatogenesis (FIG. 9). SHP2 immunoreactivity was detected in the nuclei of some Sertoli cells but not others indicating that tamoxifen induced knock out of SHP2 in Sertoli cells was less efficient than that observed for germ cells. SHP2 staining was also detected in the Sertoli cell cytoplasm of Ptpn11^{Δ/Δ} mice □along the basement membrane and surrounding germ cells.

To determine whether SHP2 is expressed in SSCs and undifferentiated spermatogonia in the testis, we assessed SHP2 co-expression with the PLZF transcription factor (Zbtb16), which is a marker for these cell types (Buaas et al., 2004; Costoya et al., 2004). In testes sections from P15 wild type mice that have a high proportion of undifferentiated spermatogonia cells, we found that SHP2 was co-expressed in most PLZF positive cells (FIG. 2B). We also found that SHP2 is co-expressed with the more rare PLZF positive cells in testis cross sections from adult wild type mice (FIG. 2C). Further studies were performed in cultures of mouse germ line stem cells (GS cells). These cultures consist of spermatogonial stem cells (SSCs) as well as undifferentiated spermatogonia derived from SSCs that are supported by mouse embryotic fibroblast feeder cells (Kanatsu-Shinohara et al., 2005; Kaucher et al., 2012; Kubota et al., 2004; Oatley et al., 2010). SHP2 was co-expressed with all PLZF positive cells in the GS cell culture indicating that SHP2 is expressed in SSCs and undifferentiated spermatogonia (FIG. 2D). In addition, there were some SHP2 positive cells that did not express PLZF suggesting that these germ cells were more differentiated. These observations are consistent with the hypothesis that SHP2 regulates SSC survival, renewal, and/or production of undifferentiated spermatogonia.

SHP2 is Required to Replenish Germ Cells.

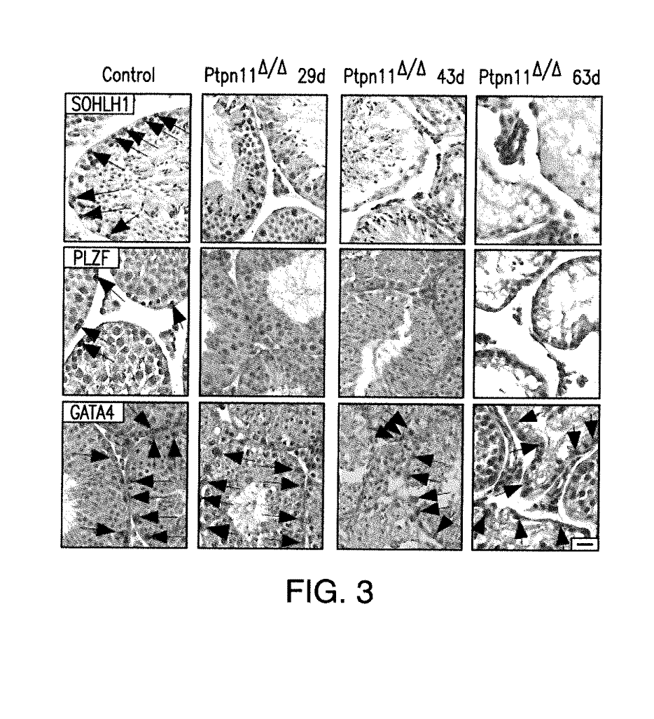

To identify the first stages of germ cell development affected by SHP2 knock out, testis cross sections from Ptpn11^{Δ/Δ} mice were probed with antisera against SOHLH1 or PLZF. These studies showed that PLZF and SOHLH1 positive cells were not detected in disrupted tubules 29 or 43 days after SHP2 knock out and no germ cells were detected at the 63 day time point (FIG. 3). The staining of GATA4 positive Sertoli cells did not change in the disrupted tubules. The lack of SOHLH1 and PLZF positive cells detected in disrupted tubules 29, 43 or 63 days after tamoxifen treatment suggests that the SSC pool is depleted in the absence of SHP2.

The Production of Germ Cell Specific SHP2 Knock Out Mice.

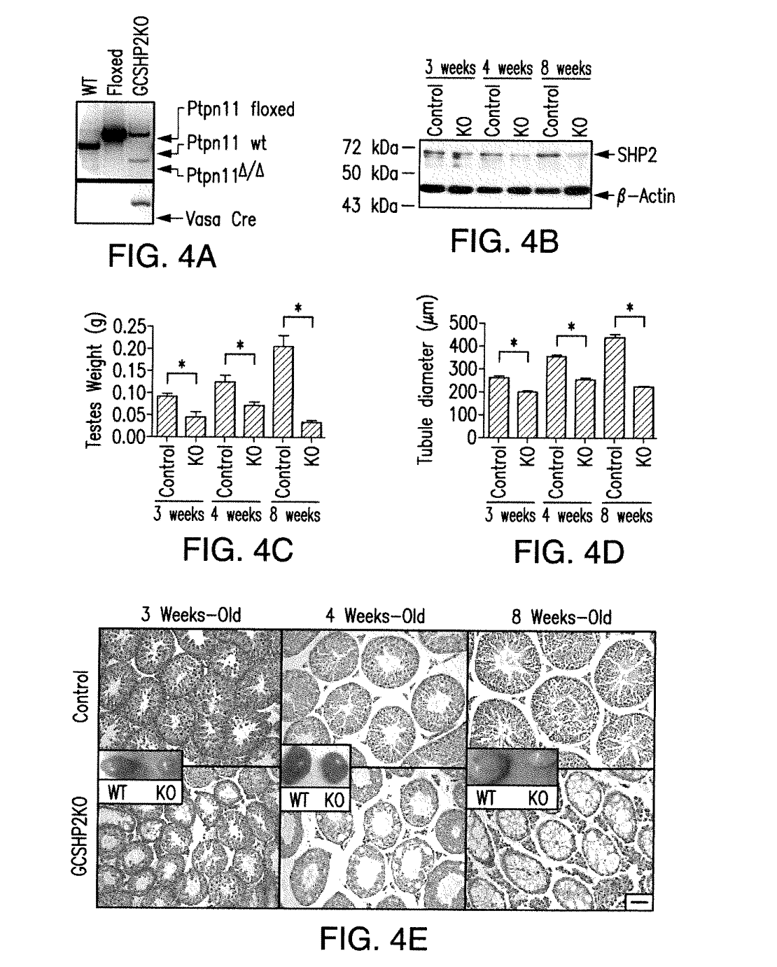

Studies of the Ptpn11^{Δ/Δ} mice indicate that SHP2 is required to maintain fertility. However, the global knockout of SHP2 does not provide information regarding whether the lack of germ cell replenishment and cell attachment defects in the absence of SHP2 are due to SHP2 actions in SSCs or in Sertoli cells that are an essential component of the SSC niche. To identify the functions of SHP2 specific to germ cells, Ptpn11^{fl/fl} mice were mated with mice expressing Cre recombinase driven by the Vasa (also known as Ddx4) promoter, which causes Cre to be expressed specifically in germ cells beginning on fetal day 15 (Gallardo et al., 2007). Genotypic analysis of progeny identified mice having Ptpn11 genes with exon 4 deleted (Ptpn11^{Δ/Δ}) and thus were germ cell specific SHP2 knock out (GCSHP2KO) mice (FIG. 4A). Western blot analysis showed that SHP2 levels decreased in GCSHP2KO mice 4 and 8 weeks after birth (FIG. 4B).

The weights of GCSHP2KO testes 3, 4 and 8 weeks after birth were reduced 56%, 46% and 80%, respectively in comparison to control littermates. Low magnification analysis of testis sections from GCSHP2KO mice revealed a progressive loss of germ cells (FIG. 4C). Diameters of the seminiferous tubules from 3-, 4- and 8-week old GCSHP2 mice were reduced by 24%, 39% and 51%, respectively (FIG. 4D). In contrast to the mosaic of disrupted tubules observed in the Ptpn11^{Δ/Δ} mice, spermatogenesis in GCSHP2KO mice was disrupted in nearly all seminiferous tubules. By eight weeks after birth, GCSHP2KO mice totally lacked germ cells in all tubule cross-sections. These data indicate that SHP2 expression in germ cells is essential to maintain the germ line. In 8 week-old GCSHP2KO mice, we also observed Leydig cell hyperplasia between the seminiferous tubules, which has been reported to occur transiently due to the deficiencies in spermatogenesis (Russell et al., 2001).

SHP2 is Required for the Production of Undifferentiated Spermatogonia.

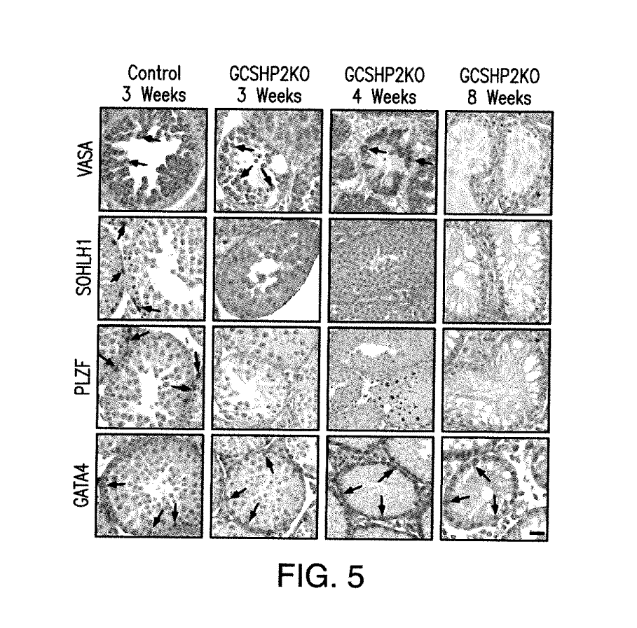

Immunohistochemistry studies performed on testes from GCSHP2KO mice 3 weeks after birth revealed that spermatocytes and spermatids were present, but Vasa positive cells were not detected on the basement membrane indicating that spermatogonia and preleptotene spermatocytes were absent (FIG. 5, top row). By 4 weeks after birth germ cells in the GCSHP2KO mice had progressed to become round spermatids and elongated spermatids but less mature germ cells from spermatogonia to pachytene spermatocytes were absent. By 8 weeks no Vasa positive germ cells were present. Probing with SOHLH1 antisera (FIG. 5, second row) or PLZF antisera (FIG. 5, third row) identified differentiated and undifferentiated spermatogonia, respectively on the basement membrane of control mouse testis but these cells were absent 3, 4 and 8 weeks after birth in GCSHP2KO mice. In contrast, staining of Sertoli cells with antiserum against GATA4 was similar in control testes and GCSHP2KO testes at all developmental ages (FIG. 5, bottom row).

The inability to replenish germ cells in GCSHP2KO mice indicates that SHP2 is essential for the survival, renewal or differentiation of SSCs. The presence of spermatocytes and spermatids in GCSHP2KO mice 3 and 4 weeks after birth demonstrates that the elimination of SHP2 specifically from germ cells did not interfere with the production of differentiated spermatogonia from gonocytes in the first wave of spermatogenesis. Also, the loss of SHP2 did not affect the survival or development of germ cells once the differentiated spermatogonia are established.

SHP2 is Required for the Self-Renewal and/or Survival of SSCs.

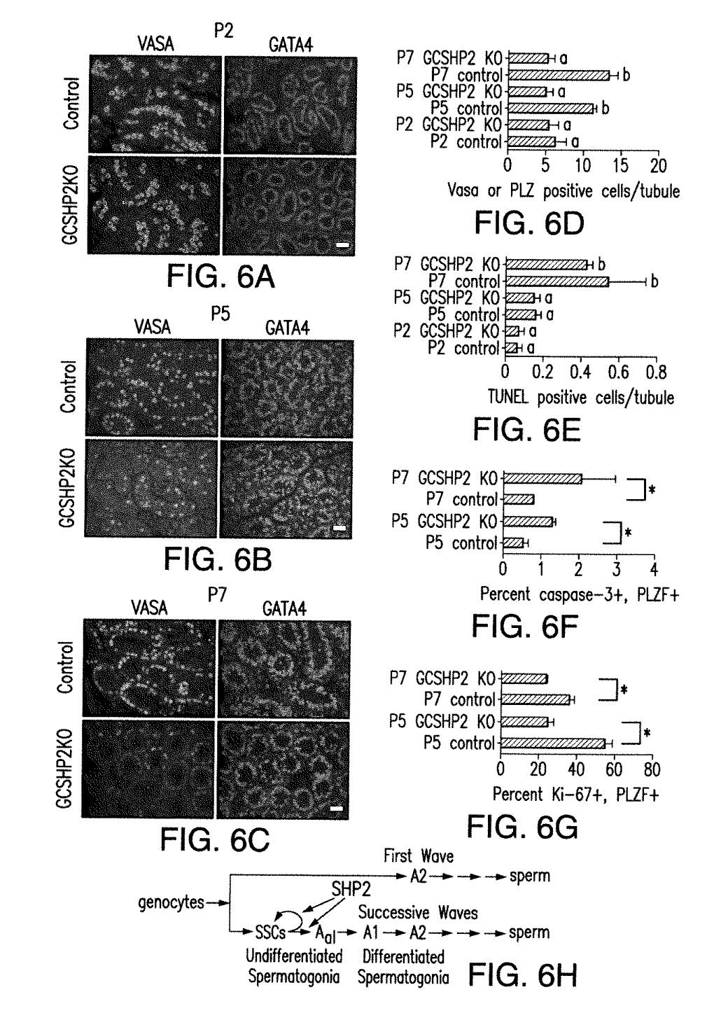

To determine whether SSCs and undifferentiated spermatogonia were absent in GCSHP2KO mice because the knock out of SHP2 caused the loss of gonocyte precursor cells, testis sections from post natal day 2 (P2) GCSHP2 mice were probed with antisera against Vasa. These studies indicated that the distribution and number of gonocytes was similar in control and GCSHP2KO mice (FIG. 6A, 6D). Thus, the lack of SHP2 does not cause the loss of the SSC precursors and SHP2 must be required at a later stage in germ cell development.

Gonocytes reenter the cell cycle on P3 and give rise to SSCs from P3.5 to P5 (Bellve et al., 1977; Yoshida et al., 2006). We compared the relative numbers of SSCs and any undifferentiated spermatogonia that were present in control and GCSHP2KO mice at P5 by probing testis tissue sections with PLZF antiserum. As expected, the proliferation of gonocytes, SSCs and undifferentiated spermatogonia in control mice increased the number of PLZF positive cells at P5 over that of the number of gonocytes (Vasa positive cells) at P2 (FIG. 6B, 6D). However, PLZF positive cells were reduced by 56% in GCSHP2 mice compared to littermate controls suggesting that there is a reduction in the number SSCs and/or undifferentiated spermatogonia in the absence of SHP2.

In control testes, the number of PLZF positive cells increased further from P5 to P7. In contrast, PLZF immunostained cells in GCSHP2KO testes did not increase and were reduced by 47% compared to control mice at P7 (FIG. 6C). The GATA4 staining of Sertoli cells was similar for control and GCSHP2KO mice at each developmental time point (FIG. 6C, 6D). TUNEL assays revealed that although apoptosis generally increased in seminiferous tubules during the P2 to P7 developmental time, course, but the overall frequency of apoptosis was relatively low (0.6 TUNEL-positive cells/tubule) (FIG. 6E). At P5 and P7, the number of apoptotic cells per tubule for GCSHP2KO and control mice were similar. However, because the number of PLZF-positive cells in GCSHP2 mice was reduced to 44% or 39% of controls, these results suggest that the remaining cells undergo apoptosis more frequently than control mice. This idea was confirmed by further analysis showing that the percentage of PLZF-positive cells in GCSHP2KO mice that were also positive for the cleaved caspase-3 marker of apoptosis was elevated by 2.5- and 2.7-fold at P5 and P7, respectively (FIG. 6F). As suggested by the TUNEL results, the frequency of apoptotic PLZF-positive cells was low in GCSHP2KO mice (2%) and did not reconcile with the 56%-61% decrease in PLZF-positive cells observed in GCSHP2KO mice. In contrast, proliferation of PLZF-positive cells as assayed by Ki-67 immunostaining was reduced by 52% in GCSHP2 mice at P5. Two days later (P7) the number of proliferating PLZF-positive cells in control mice was 35% lower, but in GCSHP2KO mice there was a further 33% decrease in PLZF-positive cells compared to control littermates (FIG. 6G). Together, these data suggest that the decrease in PLZF positive cells in GCSHP2KO mice is predominantly due to the failure of SSCs to self-renew or proliferate resulting in the inability to replenish undifferentiated spermatogonia (although apoptosis may contribute to the elimination of SSCs and undifferentiated spermatogonia) (FIG. 6H).

SHP2 Activity Supports the Attachment of SSCs to Supporting Cells, SSC Proliferation and Growth Factor Signaling Required for Renewal and Proliferation.

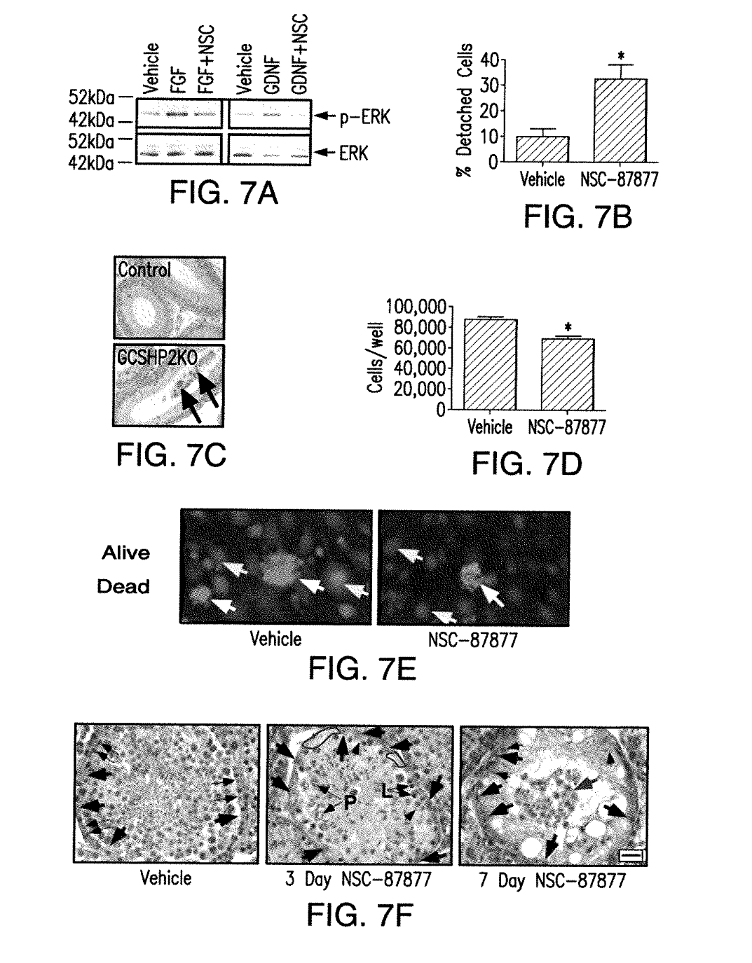

To obtain additional information regarding the mechanisms by which SHP2 acts to maintain the production of germ cells, we studied GS cell cultures. We tested whether SHP2 mediated the intracellular signals originating from FGF and GDNF growth factors that are required to maintain the survival, renewal, and proliferation of cultured GS cells and SSCs in vivo (Braydich-Stolle et al., 2007; Kubota et al., 2004; Meng et al., 2000; Ryu et al., 2005; Savitt et al., 2012). We focused on SHP2 regulation of ERK kinase that supports the renewal and proliferation of SSCs (He et al., 2008; Ishii et al., 2012; Lee et al., 2007). GS cell cultures were treated with FGF (20 ng/ml) or GDNF (20 ng/ml) for 15 min in the absence and presence of the SHP2 selective inhibitor NSC-87877 (50 μM) and then isolated from their feeder cells. Addition of FGF or GDNF increased the phosphorylation (activation) of ERK as expected (FIG. 7A). However, pretreatment with NSC-878777 inhibited the FGF and GDNF-mediated phosphorylation of ERK. These data suggest that SHP2 activity is required to transmit intracellular signals within SSCs that are required for renewal and survival. Addition of NSC-87877 (50 μM) to the GS cell cultures for 5 days resulted in a 3-fold increase in the number of GS cells that were detached from the feeder cells (FIG. 7B). This result is consistent with our hypothesis that SHP2 is required for GS cells to attach to their niche. Furthermore, we observed that prematurely released germ cells were present in the epididymis of GCSHP2KO mice 3 weeks after birth, which is consistent with the hypothesis that SHP2 activity in germ cells is required to maintain attachment to Sertoli cells (FIG. 7C).

Further studies found that a 5 day treatment with NSC-87877 caused the number of GS cells to be reduced by 22% (FIG. 7D). Cell death in the GS cultures was similarly low in the absence and presence of NSC-87877 (FIG. 7E). The magnitude of the NSC-87877-mediated decrease in GS cells is consistent with the inability of the less abundant SSCs to self-renew or proliferate which causes a decrease in cell multiplication. However, we cannot rule out the possibility that NSC-87877 specifically mediated the death of SSCs that make up a small portion of the cell population.

Injection (ip) of NSC-87877 (5 mg/kg) into adult mice daily for 3 or 7 days resulted in the absence of spermatogonia 15 days later in tubules at the periphery of the testes (FIG. 7F). These results provide further evidence that SHP2 activity is required for SSCs to replenish germ cells. The NSC-87877 injections also caused further disruption of spermatogenesis that was similar to that observed for Ptpn11^{Δ/Δ} mice including the mis-localization and mis-orientation of elongated spermatids. Furthermore, the populations of spermatocytes and spermatids were reduced. In some tubule cross sections from mice treated with the SHP2 inhibitor for 7 days, detached germ cells were observed and nearly all germ cells were absent.

### 6.3 Discussion

Using three complimentary mouse models in which SHP2 expression is eliminated either in all cells or only germ cells, we found that SHP2 is essential to complete the initial step of spermatogenesis, which is the production of undifferentiated spermatogonia from SSCs. In the absence of SHP2, undifferentiated spermatogonia are not produced but germ cells beyond this stage of development are capable of completing the process of spermatogenesis.

The inability to produce undifferentiated spermatogonia is not due to the death of gonocyte precursors of SSC because the elimination of SHP2 specifically in germ cells did not alter the number of gonocytes that were present 2 days after birth. We cannot rule out the possibility that SHP2 contributes to the transition of gonocytes into SSCs between P2 and P5. However, ablation of SHP2 in adult Ptpn11^{Δ/Δ} mice prevents the replenishment of undifferentiated spermatogonia well after all gonocytes have been converted to SSCs indicating that the lack of undifferentiated spermatogonia must be due to a defect in SSCs. In the GCSHP2KO mouse, the decrease in PLZF positive cells (SSCs and undifferentiated spermatogonia) on P5 and P7 occurred in the absence of decreased cell survival. This result suggests that the loss of SHP2 does not cause the death of undifferentiated spermatogonia but supports the hypothesis that ablation of SHP2 rapidly ceases germ cell renewal or proliferation. Although some undifferentiated spermatogonia can be produced in GCSHP2KO mice by P5 and P7, the lack of SHP2 totally abolishes germ cell production prior to 3 weeks after birth. These findings suggest that in the absence of SHP2, SSCs can divide to produce only spermatogonia committed to terminal differentiation and thus deplete the pool of SSCs. The remaining SSCs are unable to self-renew or die in the process of self-renewal.

Studies of GS cell cultures support the hypothesis that SHP2 is required for SSC self-renewal as inhibition of SHP2 reduced the number of cells present in the GS cultures by 22% without any detectable decrease in cell survival. The relatively small but significant reduction in the number of GS cells after 5 days of SHP2 inhibitor treatment is consistent with blocking the proliferation or self renewal of the more rare SSCs (<10% of the culture) that are needed to produce transit amplifying undifferentiated spermatogonia. The smaller magnitude of decrease for cultured GS cells compared to PLZF positive cells in vivo after the loss of SHP2 activity also is consistent with the slower proliferation of GS cells (5.6 day doubling time) (Kubota et al., 2004) versus that of undifferentiated spermatogonia in vivo (30 to 70 h) (de Rooij and Russell, 2000; Yoshida et al., 2007).

The absence of spermatogonia on the basement membrane of seminiferous tubules 15 days after injection of mice with the SHP2 inhibitor NSC-87877 further supports the hypothesis that SHP2 is required for SSCs to replenish germ cells. Inhibition of SHP2 produced a dramatic disruption of spermatogenesis that was similar to that observed after knock out of SHP2 in the Ptpn11^{Δ/Δ} □ mice including the detachment of meiotic and post-meiotic germ cells as well as mis-localization and retention of elongated spermatids. These data suggest that SHP2 performs functions in Sertoli cells (and perhaps other cells) in addition to germ cells that are required to support spermatogenesis. The elimination of SSCs and differentiated germ cells also suggests inhibitors of SHP2 or SHP2 targets could be used to permanently sterilize animals as a mechanism to humanely solve challenges associated with overpopulation.

The role of SHP2 in replenishing germ cells from SSCs is reminiscent of SHP2 functions in other stem cells. Deletion of the SHP2 gene in hematopoietic stem cells (HSCs) disrupts their quiescent state, and self-renewal while increasing apoptosis resulting in a severe reduction in mature blood cells (Chan et al., 2011; Zhu et al., 2011). Similarly, the survival of trophoblast stem cells is dependent upon SHP2 (Yang et al., 2006); whereas SHP2 deletion in neuronal stem cells impairs their proliferation and differentiation resulting in lethality (Ke et al., 2007).

The progressive loss of germ cells and the resulting Sertoli cell only phenotype that was found after elimination of SHP2 expression has been reported in mice engineered to lack expression of spermatogonial stem cell markers such as PLZF, ETV5 FOXO1 and Sin3a (Chen et al., 2005; Gallagher et al., 2013; Goertz et al., 2011; Schlesser et al., 2008). Mice lacking a single allele of GDNF, a growth factor required to SSC renewal, also show a similar phenotype (Meng et al., 2000) and GDNF is known to activate SHP2 in other cells (Perrinjaquet et al., 2010; Willecke et al., 2011). FGF that acts in concert with GDNF to maintain the pool of SSCs, also mediates its signaling through SHP2 in a variety of cell types (Cai et al., 2010; Hatanaka et al., 2012; Ishii et al., 2012; Mansukhani et al., 2000; Pan et al., 2008). Additionally, the ETV5 and FOXO1 transcription factors that are required to maintain the SSC pool (Goertz et al., 2011; Willecke et al., 2011) are known downstream targets of SHP2 (Willecke et al., 2011; Zhang et al., 2009).

Interestingly, the progressive loss of germ cells occurs more rapidly after the loss of SHP2 in comparison to other factors that are essential for SCC maintenance. This more dramatic phenotype of the SHP2 deficient mouse models may be due to SHP2 being a mediator of both GDNF and FGF signaling as well as a regulator of ETV5, FOXO1 and likely other factors required for SSC self-renewal and/or survival. Thus, without being bound by theory, we propose that SHP2 may be a central nexus and rheostat for intracellular signaling pathways in SSCs that are essential for the fine-tuning of signals required for the self-renewal and the production of undifferentiated spermatogonia. Supporting this hypothesis was our finding that inhibition of SHP2 activity by NSC-87877 in cultured GS cells decreased FGF and GDNF mediated activation of ERK.

Although SHP2 is required for the production of new germ cells, the transient presence of pachytene spermatocytes and spermatids in Ptpn11^{Δ/Δ} mice showed that germ cells that were beyond the undifferentiated spermatogonia stage of development at the time of SHP2 knock out were able to continue their maturation. Thus, a “final wave” of spermatogenesis continues after SHP2 knock out. In the GCSHP2KO mice, by 3 and 4 weeks after birth, germ cells that were less mature than pachytene spermatocytes were absent but pachytene spermatocytes and spermatids were produced. These results indicate that the first wave of spermatogenesis occurs after knock out of SHP2. The production of the first wave provides further evidence that gonocytes in GCSHP2KO mice are able to produce differentiated spermatids as usual 3 to 5 days after birth and confirms that the lack of SHP2 expression only affects the survival of germ cells prior to the differentiated spermatogonia stage of development.

NSC-87877-mediated inhibition of SHP2 caused a 3-fold increase in the number of GS cells that were detached from their feeder cells. This result raises the possibility that SHP2 is required to maintain SSCs in their niche and that release from the niche may contribute to decreased survivability of SSCs or remove the SSCs from the signals that are required for their renewal, proliferation, and/or ability to produce undifferentiated spermatogonia. Presently, it is not known whether the detachment of SSCs is caused by the inhibition of SHP2 in SSCs or their supporting cells.

Further evidence for SHP2 regulation of cellular attachments was provided by the Ptpn11^{Δ/Δ} mouse model in which the elimination of SHP2 resulted in the release of immature germ cells that were found in the epididymis. Ptpn11^{Δ/Δ} mice can lack SHP2 in both Sertoli cells and germ cells. From this model alone, it cannot be determined whether the release of immature germ cells occurs in regions of SHP2 deficient Sertoli cells or whether the ablation of SHP2 in germ cells causes their premature release. Interestingly, few Sertoli cells displayed reduced SHP2 expression indicating that tamoxifen inducible knock out of SHP2 is less efficient in Sertoli cells. Similar results were observed in Sertoli cells for tamoxifen inducible elimination of the androgen receptor using a different Cre recombinase (Willems et al., 2011). It has been suggested that Cre activity may be lower in Sertoli cells due to lower expression of the transgene or lower effectiveness of tamoxifen in Sertoli cells due to the expression of drug transporters involved in the protective function of Sertoli cells during germ cell development (Cheng and Mruk, 2012; Su et al., 2011; Willems et al., 2011).

The finding that immature germ cells are found in the epididymis of GCSHP2KO mice suggests that the loss of SHP2 specifically in germ cells is sufficient to disrupt the attachment of spermatocytes and spermatids to Sertoli cells. However, SHP2 expression is only detected prior to the differentiation of spermatogonia. It is possible that the effects of SHP2 loss in undifferentiated spermatogonia are manifest later in germ cell development or that the low levels of SHP2 in meiotic and post meiotic germ cells are sufficient for maintaining Sertoli-germ cell connections. Additional studies will be required to determine the relative contributions of SHP2 in Sertoli cells and germ cells toward maintaining germ cell attachment.

Ptpn11^{Δ/Δ} mice and mice lacking the SHP2 regulated ETV5 gene are similar in that both display mis-orientation of elongated spermatids, spermiation failure and phagocytosis of elongated spermatids by Sertoli cells (Schlesser et al., 2008) suggesting that SHP2 is part of a signaling pathway required to regulate germ cell attachment. The localization of SHP2 immunoreactivity to Sertoli-elongating spermatid attachment sites is consistent with our observations that SHP2 is a regulator of Sertoli-germ cell adhesion and is in agreement with our recent findings that constitutive activation of SHP2 disrupts Sertoli-Sertoli cell junctional complexes by mis-localization of adherens junction proteins β-catenin and N-cadherin and disruption of actin cytoskeleton (Puri and Walker, 2013).

Together, our findings show that expression of SHP2 is critical for maintaining the germ line in males. In germ cells, SHP2 mediates GDNF and FGF signals needed for the survival of SSCs and the production of the undifferentiated spermatogonia. SHP2 also functions in germ cells and the Sertoli cell to maintain cell-cell attachments that are required to maintain the niche for SSCs and developing germ cells as well as migration of elongated spermatids and the release of mature spermatozoa. The rapid and complete loss of germ cells after knock out or inhibition of SHP2 suggests that inhibitors of SHP2 could be used as sterilents to block sperm production at the initiation of spermatogenesis. Finally, these new findings may be applied to the development of therapies for Noonan and LEOPARD syndrome that have SHP2 defects and reduced fertility.

### 7. EXAMPLE 2

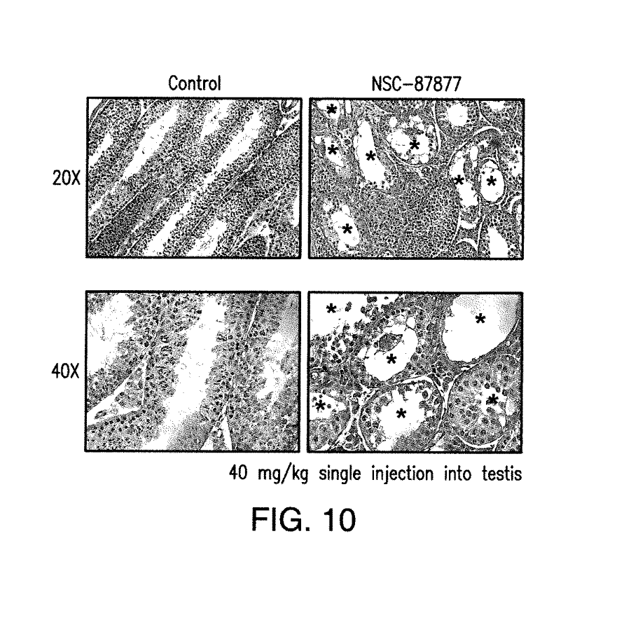

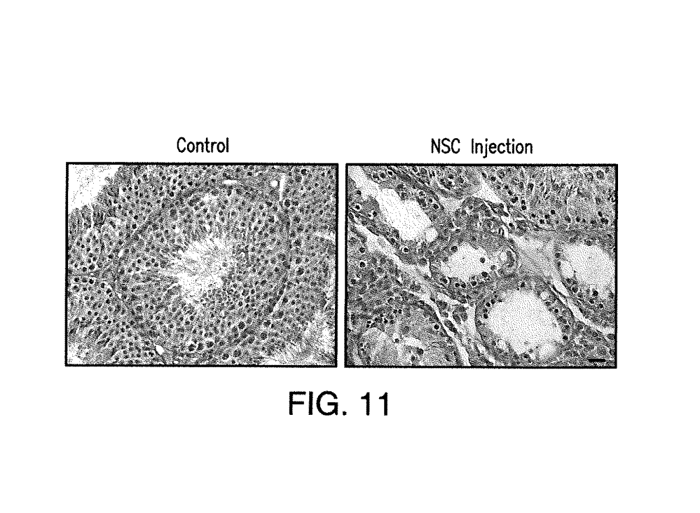

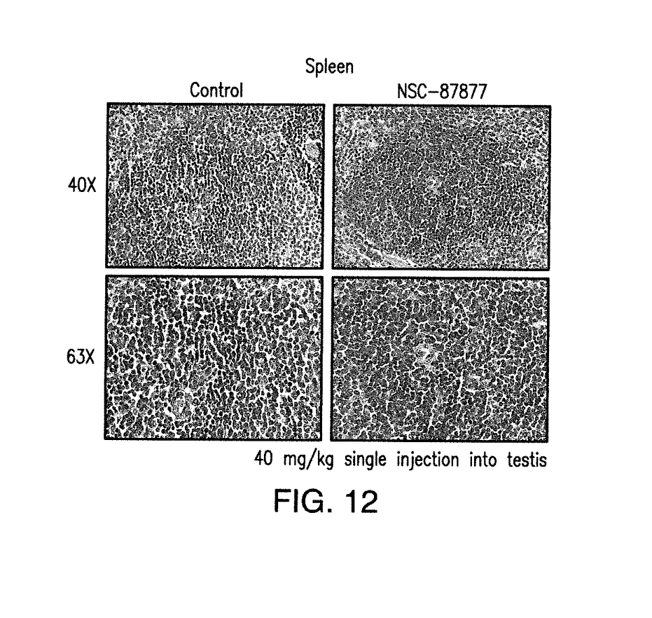

The preceding working example showed that multiple injections of SHP2 inhibitor via ip blocked sperm production. This working example relates to additional studies showing that 1 injection of the SHP2 inhibitor, at a dose of 40 mg/kg, into the adult testes of a male mouse was sufficient to disrupt spermatogenesis (sperm production) 15 days later (FIG. 10). A second study showed that 1 injection of the inhibitor at a dose of 10 mg/kg into the testes of an adult male mouse could produce long term (70 days) loss of sperm production in some regions of the testes (FIG. 11), Asterisks denote regions of the testes lacking germ cells and sperm. FIG. 12 shows that spleen tissue from the same animal was not damaged 15 days after injection of the inhibitor into testes.

### 8. REFERENCES

Arrandale, J. M., Gore-Willse, A., Rocks, S., Ren, J. M., Zhu, J., Davis, A., Livingston, J. N., and Rabin, D. U. (1996). Insulin signaling in mice expressing reduced levels of Syp. J Biol Chem 271, 21353-21358.
Bauler, T. J., Kamiya, N., Lapinski, P. E., Langewisch, E., Mishina, Y., Wilkinson, J. E., Feng, G. S., and King, P. D. (2011). Development of severe skeletal defects in induced SHP-2-deficient adult mice: a model of skeletal malformation in humans with SHP-2 mutations. Dis Model Mech 4, 228-239.
Bellve, A. R., Cavicchia, J. C., Millette, C. F., O'Brien, D. A., Bhatnagar, Y. M., and Dym, M. (1977). Spermatogenic cells of the prepuberal mouse. Isolation and morphological characterization. J Cell Biol 74, 68-85.
Braydich-Stolle, L., Kostereva, N., Dym, M., and Hofmann, M. C. (2007). Role of Src family kinases and N-Myc in spermatogonial stem cell proliferation. Dev Biol 304, 34-45.
Buaas, F. W., Kirsh, A. L., Sharma, M., McLean, D. J., Morris, J. L., Griswold, M. D., de Rooij, D. G., and Braun, R. E. (2004). Plzf is required in adult male germ cells for stem cell self-renewal. Nat Genet 36, 647-652.
Cai, Z., Feng, G. S., and Zhang, X. (2010). Temporal requirement of the protein tyrosine phosphatase Shp2 in establishing the neuronal fate in early retinal development. The Journal of neuroscience: the official journal of the Society for Neuroscience 30, 4110-4119.
Chan, G., Cheung, L. S., Yang, W., Milyavsky, M., Sanders, A. D., Gu, S., Hong, W. X., Liu, A. X., Wang, X., Barbara, M., et al. (2011). Essential role for Ptpn11 in survival of hematopoietic stem and progenitor cells. Blood 117, 4253-4261.
Chan, R. J., and Feng, G. S. (2007). PTPN11 is the first identified proto-oncogene that encodes a tyrosine phosphatase. Blood 109, 862-867. Chen, C., Ouyang, W., Grigura, V., Zhou, Q., Carnes, K., Lim, H., Zhao, G. Q., Arber, S.,
Kurpios, N., Murphy, T. L., et al. (2005). ERM is required for transcriptional control of the spermatogonial stem cell niche. Nature 436, 1030-1034.
Chen, L. et al. (2006). Mol. Pharmacol. 70(2):562-570.
Chen, L. et al. (2010). Biochem. Pharmacol. 80(6):801-810.
Cheng, C. Y., and Mruk, D. D. (2012). The blood-testis barrier and its implications for male contraception. Pharmacol Rev 64, 16-64.
Costoya, J. A., Hobbs, R. M., Barna, M., Cattoretti, G., Manova, K., Sukhwani, M., Orwig, K. E., Wolgemuth, D. J., and Pandolfi, P. P. (2004). Essential role of Plzf in maintenance of spermatogonial stem cells. Nat Genet 36, 653-659.
de Rooij, D. G., and Russell, L. D. (2000). All you wanted to know about spermatogonia but were afraid to ask. J Androl 21, 776-798.
Dolci, S., Williams, D. E., Ernst, M. K., Resnick, J. L., Brannan, C. I., Lock, L. F., Lyman, S. D., Boswell, H. S., and Donovan, P. J. (1991). Requirement for mast cell growth factor for primordial germ cell survival in culture. Nature 352, 809-811.
Edouard, T., Montagner, A., Dance, M., Conte, F., Yart, A., Parfait, B., Tauber, M., Salles, J. P., and Raynal, P. (2007). How do Shp2 mutations that oppositely influence its biochemical activity result in syndromes with overlapping symptoms?Cell Mol Life Sci 64, 1585-1590.
Gallagher, S. J., Kofman, A. E., Huszar, J. M., Dannenberg, J. H., DePinho, R. A., Braun, R. E., and Payne, C. J. (2013). Distinct requirements for Sin3a in perinatal male gonocytes and differentiating spermatogonia. Dev Biol 373, 83-94.
Gallardo, T., Shirley, L., John, G. B., and Castrillon, D. H. (2007). Generation of a germ cell-specific mouse transgenic Cre line, Vasa-Cre. Genesis 45, 413-417.
Gauthier, A. S., Furstoss, O., Araki, T., Chan, R., Neel, B. G., Kaplan, D. R., and Miller, F. D. (2007). Control of CNS cell-fate decisions by SHP-2 and its dysregulation in Noonan syndrome. Neuron 54, 245-262.
Godin, I., Deed, R., Cooke, J., Zsebo, K., Dexter, M., and Wylie, C. C. (1991). Effects of the steel gene product on mouse primordial germ cells in culture. Nature 352, 807-809.
Goertz, M. J., Wu, Z., Gallardo, T. D., Hamra, F. K., and Castrillon, D. H. (2011). Foxo1 is required in mouse spermatogonial stem cells for their maintenance and the initiation of spermatogenesis. J Clin Invest 121, 3456-3466.
Grossmann, K. S., Rosario, M., Birchmeier, C., and Birchmeier, W. (2010). The tyrosine phosphatase Shp2 in development and cancer. Adv Cancer Res 106, 53-89.
Hatanaka, K., Lanahan, A. A., Murakami, M., and Simons, M. (2012). Fibroblast Growth Factor Signaling Potentiates VE-Cadherin Stability at Adherens Junctions by Regulating SHP2. PLoS One 7, e37600.
He, Z., Jiang, J., Kokkinaki, M., Golestaneh, N., Hofmann, M. C., and Dym, M. (2008). Gdnf upregulates c-Fos transcription via the Ras/Erkl/2 pathway to promote mouse spermatogonial stem cell proliferation. Stem Cells 26, 266-278.
Hellmuth, K. et al. (2008) Proc. Natl. Acad. Sci. U.S.A. 105(20):7275-7280.
Ishii, K., Kanatsu-Shinohara, M., Toyokuni, S., and Shinohara, T. (2012). FGF2 mediates mouse spermatogonial stem cell self-renewal via upregulation of Etv5 and Bcl6b through MAP2K1 activation. Development 139, 1734-1743.
Jorge, A. A., Malaquias, A. C., Arnhold, I. J., and Mendonca, B. B. (2009). Noonan syndrome and related disorders: a review of clinical features and mutations in genes of the RAS/MAPK pathway. Horm Res 71, 185-193.
Kanatsu-Shinohara, M., Miki, H., Inoue, K., Ogonuki, N., Toyokuni, S., Ogura, A., and Shinohara, T. (2005). Long-term culture of mouse male germline stem cells under serum- or feeder-free conditions. Biol Reprod 72, 985-991.
Kandadi, M. R., Stratton, M. S., and Ren, J. (2010). The role of Src homology 2 containing protein tyrosine phosphatase 2 in vascular smooth muscle cell migration and proliferation. Acta Pharmacol Sin 31, 1277-1283.
Kaucher, A. V., Oatley, M. J., and Oatley, J. M. (2012). NEUROG3 is a critical downstream effector for STAT3-regulated differentiation of mammalian stem and progenitor spermatogonia. Biol Reprod 86, 164, 161-111.
Ke, Y., Zhang, E. E., Hagihara, K., Wu, D., Pang, Y., Klein, R., Curran, T., Ranscht, B., and Feng, G. S. (2007). Deletion of Shp2 in the brain leads to defective proliferation and differentiation in neural stem cells and early postnatal lethality. Mol Cell Biol 27, 6706-6717.
Kubota, H., Avarbock, M. R., and Brinster, R. L. (2004). Growth factors essential for self-renewal and expansion of mouse spermatogonial stem cells. Proc Natl Acad Sci USA 101, 16489-16494.
Lee, J., Kanatsu-Shinohara, M., Inoue, K., Ogonuki, N., Miki, H., Toyokuni, S., Kimura, T., Nakano, T., Ogura, A., and Shinohara, T. (2007). Akt mediates self-renewal division of mouse spermatogonial stem cells. Development 134, 1853-1859.
Liu, S. et al. (2011). Chem. Biol. 18:101-110.
Mansukhani, A., Bellosta, P., Sahni, M., and Basilico, C. (2000). Signaling by fibroblast growth factors (FGF) and fibroblast growth factor receptor 2 (FGFR2)-activating mutations blocks mineralization and induces apoptosis in osteoblasts. J Cell Biol 149, 1297-1308.
Meng, X., Lindahl, M., Hyvonen, M. E., Parvinen, M., de Rooij, D. G., Hess, M. W., Raatikainen-Ahokas, A., Sainio, K., Rauvala, H., Lakso, M., et al. (2000). Regulation of cell fate decision of undifferentiated spermatogonia by GDNF. Science 287, 1489-1493.
Metzger, D., and Chambon, P. (2001). Site- and time-specific gene targeting in the mouse. Methods 24, 71-80.
Nagano, M., Ryu, B. Y., Brinster, C. J., Avarbock, M. R., and Brinster, R. L. (2003). Maintenance of mouse male germ line stem cells in vitro. Biol Reprod 68, 2207-2214.
Oatley, J. M., and Brinster, R. L. (2006). Spermatogonial stem cells. Methods Enzymol 419, 259-282.
Oatley, J. M., Kaucher, A. V., Avarbock, M. R., and Brinster, R. L. (2010). Regulation of mouse spermatogonial stem cell differentiation by STAT3 signaling. Biol Reprod 83, 427-433.
Pan, Y., Carbe, C., Powers, A., Zhang, E. E., Esko, J. D., Grobe, K., Feng, G. S., and Zhang, X. (2008). Bud specific N-sulfation of heparan sulfate regulates Shp2-dependent FGF signaling during lacrimal gland induction. Development 135, 301-310.
Perrinjaquet, M., Vilar, M., and Ibanez, C. F. (2010). Protein-tyrosine phosphatase SHP2 contributes to GDNF neurotrophic activity through direct binding to phospho-Tyr687 in the RET receptor tyrosine kinase. J Biol Chem 285, 31867-31875.
Puri, P. (2012). “SHP2 Signaling in Spermatogenesis and Male Fertility.” Seminars for the Magee Womens Research Institute May 24, 2012.
Puri, P. (2012) “The SHP2 Tyrosine Phosphatase is Essential for Spermatogenesis and Male Fertility.” Society for the Study of Reproduction (SSR) 45th Annual Meeting State College, Pa., 12-15 Aug. 2012.
Puri, P., and Walker, W. H. (2013). The Tyrosine Phosphatase SHP2 Regulates Sertoli Cell Junction Complexes. Biol Reprod 88, 59, 51-11.
Puri et al., (2014). The transition from stem cell to progenitor spermatogonia and male fertility requires the SHP2 protein tyrosine phosphatase. Stem Cells 32(3), 741-753.
Russell, L. D., Saxena, N. K., and Turner, T. T. (1989). Cytoskeletal involvement in spermiation and sperm transport. Tissue & cell 21, 361-379.
Russell, L. D., Warren, J., Debeljuk, L., Richardson, L. L., Mahar, P. L., Waymire, K. G., Amy, S. P., Ross, A. J., and MacGregor, G. R. (2001). Spermatogenesis in Bclw-deficient mice. Biol Reprod 65, 318-332.
Ruzankina, Y., Pinzon-Guzman, C., Asare, A., Ong, T., Pontano, L., Cotsarelis, G., Zediak, V. P., Velez, M., Bhandoola, A., and Brown, E. J. (2007). Deletion of the developmentally essential gene ATR in adult mice leads to age-related phenotypes and stem cell loss. Cell Stem Cell 1, 113-126.
Ryu, B. Y., Kubota, H., Avarbock, M. R., and Brinster, R. L. (2005). Conservation of spermatogonial stem cell self-renewal signaling between mouse and rat. Proc Natl Acad Sci USA 102, 14302-14307.
Sadate-Ngatchou, P. I., Payne, C. J., Dearth, A. T., and Braun, R. E. (2008). Cre recombinase activity specific to postnatal, premeiotic male germ cells in transgenic mice. Genesis 46, 738-742.
Savitt, J., Singh, D., Zhang, C., Chen, L. C., Folmer, J., Shokat, K. M., and Wright, W. W. (2012). The in vivo response of stem and other undifferentiated spermatogonia to the reversible inhibition of glial cell line-derived neurotrophic factor signaling in the adult. Stem Cells 30, 732-740.
Saxton, T. M., Henkemeyer, M., Gasca, S., Shen, R., Rossi, D. J., Shalaby, F., Feng, G. S., and Pawson, T. (1997). Abnormal mesoderm patterning in mouse embryos mutant for the SH2 tyrosine phosphatase Shp-2. Embo J 16, 2352-2364.
Schlesser, H. N., Simon, L., Hofmann, M. C., Murphy, K. M., Murphy, T., Hess, R. A., and Cooke, P. S. (2008). Effects of ETV5 (ets variant gene 5) on testis and body growth, time course of spermatogonial stem cell loss, and fertility in mice. Biol Reprod 78, 483-489.
Shupe, J. et al. (2011). Regulation of Sertoli-germ cell adhesion and sperm release by FSH and nonclasical testosterone signalling. Mol. Endocrinol. 25(2):238-252.
Su, L., Mruk, D. D., Lui, W. Y., Lee, W. M., and Cheng, C. Y. (2011). P-glycoprotein regulates blood-testis barrier dynamics via its effects on the occludin/zonula occludens 1 (ZO-1) protein complex mediated by focal adhesion kinase (FAK). Proc Natl Acad Sci USA 108, 19623-19628.
Suzuki, H., Ahn, H. W., Chu, T., Bowden, W., Gassei, K., Orwig, K., and Rajkovic, A. (2012). SOHLH1 and SOHLH2 coordinate spermatogonial differentiation. Dev Biol 361, 301-312.
Tartaglia, M., Niemeyer, C. M., Fragale, A., Song, X., Buechner, J., Jung, A., Hahlen, K., Hasle, H., Licht, J. D., and Gelb, B. D. (2003). Somatic mutations in PTPN11 in juvenile myelomonocytic leukemia, myelodysplastic syndromes and acute myeloid leukemia. Nat Genet 34, 148-150.
Tegelenbosch, R. A., and de Rooij, D. G. (1993). A quantitative study of spermatogonial multiplication and stem cell renewal in the C3H/101 F1 hybrid mouse. Mutat Res 290, 193-200.
Walker, W H (2010). Non-classical actions of testosterone and spermatogenesis. Philo. Trans. R. Soc. Lond. B. Biol. Sci. 365(1546), 1557-1569.
Walker, W. H. (2011). Testosterone signaling and the regulation of spermatogenesis. Spermatogenesis 1(2), 116-120.
Willecke, R., Heuberger, J., Grossmann, K., Michos, O., Schmidt-Ott, K., Walentin, K., Costantini, F., and Birchmeier, W. (2011). The tyrosine phosphatase Shp2 acts downstream of GDNF/Ret in branching morphogenesis of the developing mouse kidney. Dev Biol 360, 310-317.
Willems, A., De Gendt, K., Deboel, L., Swinnen, J. V., and Verhoeven, G. (2011). The development of an inducible androgen receptor knockout model in mouse to study the postmeiotic effects of androgens on germ cell development. Spermatogenesis 1, 341-353.
Won, K J et al. (2011) J. Pharmacol. Sci. 115(2):164-175. Wood, M. A. et al. (2011). Upstream stimulatory factor induces Nr5a1 and Shbg gene expression during the onset of rat Sertoli cell differentiation. Biol Reprod. 85(5), 965-976.
Wu et al., United States Patent Publication No. 20120034186 (U. S. Ser. No. 13/274,699) Xu, D., and Qu, C. K. (2008). Protein tyrosine phosphatases in the JAK/STAT pathway. Front Biosci 13, 4925-4932.
Yang, W., Klaman, L. D., Chen, B., Araki, T., Harada, H., Thomas, S. M., George, E. L., and Neel, B. G. (2006). An Shp2/SFK/Ras/Erk signaling pathway controls trophoblast stem cell survival. Dev Cell 10, 317-327.
Yoshida, S., Sukeno, M., and Nabeshima, Y. (2007). A vasculature-associated niche for undifferentiated spermatogonia in the mouse testis. Science 317, 1722-1726.
Yoshida, S., Sukeno, M., Nakagawa, T., Ohbo, K., Nagamatsu, G., Suda, T., and Nabeshima, Y. (2006). The first round of mouse spermatogenesis is a distinctive program that lacks the self-renewing spermatogonia stage. Development 133, 1495-1505.
Yu, Z. H. et al. (2011) Bioorg. Med. Chem. Lett. 21(14):4238-4242.
Zhang, E. E., Chapeau, E., Hagihara, K., and Feng, G. S. (2004). Neuronal Shp2 tyrosine phosphatase controls energy balance and metabolism. Proc Natl Acad Sci USA 101, 16064-16069.
Zhang, S. S., Hao, E., Yu, J., Liu, W., Wang, J., Levine, F., and Feng, G. S. (2009). Coordinated regulation by Shp2 tyrosine phosphatase of signaling events controlling insulin biosynthesis in pancreatic beta-cells. Proc Natl Acad Sci USA 106, 7531-7536.
Zhu, H. H., Ji, K., Alderson, N., He, Z., Li, S., Liu, W., Zhang, D. E., Li, L., and Feng, G. S. (2011). Kit-Shp2-Kit signaling acts to maintain a functional hematopoietic stem and progenitor cell pool. Blood 117, 5350-5361.
United States Patent Application Publication No. US20080176309, entitled “Inhibition of Shp2/PTPN11 Protein Tyrosine Phosphatase by NSC-87877, NSC-117199 and Their Analogs”

Various publications are cited herein, the contents of which are hereby incorporated by reference in their entireties.

What is claimed is:

1. A method of reducing fertility in a subject comprising administering, to the subject, an effective amount of a SHP2 inhibitor.

2. The method of claim 1, where the subject is a dog.

3. The method of claim 1, where the subject is a cat.

4. The method of claim 1, where the subject is a human.

5. The method of claim 1, where the SHP2 inhibitor is NSC-87877.

6. A method of reducing spermatogenesis in a subject comprising administering, to the subject, an effective amount of a SHP2 inhibitor.

7. The method of claim 6, where the subject is a dog.

8. The method of claim 6, where the subject is a cat.

9. The method of claim 6, where the subject is a human.

10. The method of claim 6, where the SHP2 inhibitor is NSC-87877.

11. A SHP2 inhibitor, for use in reducing fertility in a subject.

12. The SHP2 inhibitor of claim 11, where the subject is a dog.

13. The SHP2 inhibitor of claim 11, where the subject is a cat.

14. The SHP2 inhibitor of claim 11, where the subject is a human.

15. The SHP2 inhibitor of claim 11 which is NSC-87877.

16. A SHP2 inhibitor, for use in preparing a pharmaceutical composition for reducing fertility in a subject.

17. The SHP2 inhibitor of claim 16, where the subject is a dog.

18. The SHP2 inhibitor of claim 17, where the subject is a cat.

19. The SHP2 inhibitor of claim 18, where the subject is a human.

20. The SHP2 inhibitor of claim 16 which is NSC-87877.

21. A SHP2 inhibitor, for use in reducing spermatogenesis in a subject.

22. The SHP2 inhibitor of claim 21, where the subject is a dog.

23. The SHP2 inhibitor of claim 21, where the subject is a cat.

24. The SHP2 inhibitor of claim 21, where the subject is a human.

25. The SHP2 inhibitor of claim 21 which is NSC-87877.

26. A SHP2 inhibitor, for use in preparing a pharmaceutical composition for reducing spermatogenesis in a subject.

27. The SHP2 inhibitor of claim 26, where the subject is a dog.

28. The SHP2 inhibitor of claim 27, where the subject is a cat.

29. The SHP2 inhibitor of claim 28, where the subject is a human.

30. The SHP2 inhibitor of claim 26 which is NSC-87877.
